(19) United States
(12) Patent Application Publication
(10) Pub. No.: US 2020/0338178 A1
(43) Pub. Date: Oct. 29, 2020

(54) MALARIAL VACCINATION METHODS AND REGIMENS

(71) Applicant: UNIVERSITY OF WASHINGTON, Seattle, WA (US)

(72) Inventors: Sean C. MURPHY, Seattle, WA (US); Bradley C. Stone, Seattle, WA (US)

(21) Appl. No.: 16/946,861

(22) Filed: Jul. 9, 2020

Related U.S. Application Data
(63) Continuation of application No. PCT/US2019/013114, filed on Jan. 10, 2019.
(60) Provisional application No. 62/615,755, filed on Jan. 10, 2018.

(51) Int. Cl. A61K 39/015; A61K 39/39

(52) U.S. Cl. CPC A61K 39/015; A61K 2039/545; A61K 39/39

(57) ABSTRACT

Compositions, methods, and regimens for vaccinating subjects against malaria parasites are provided herein. The compositions include first and second compositions having priming and/or trapping components. The priming components include, but are not limited to, DNA or RNA polynucleotides, and the trapping components include, but are not limited to, DNA or RNA polynucleotides, one or more attenuated sporozoites and/or other liver-specific antigens as viral or other formulations. Following administration of one or more of the compositions provided herein using one or more of the vaccination regimens and/or methods, high levels of liver resident memory CD8+ T cells are induced in the subjects leading to protection against sporozoite challenge.

### PRIORITY CLAIM

This application is a continuation of International Application No. PCT/US2019/013114, filed Jan. 10, 2019, which claims priority to U.S. Provisional Application No. 62/615,755 filed Jan. 10, 2018, the entire contents of both of which is incorporated herein by reference and relied upon in its entirety.

### BACKGROUND

Malaria parasites cause approximately 250 million infections and 429,000 deaths annually, mostly in children under five years old (WHO). Efforts are underway to target the pre-erythrocytic (e.g., sporozoite and liver stages), erythrocytic and gametocyte stages. The pre-erythrocytic stage is an attractive vaccine target because the number of infected cells is relatively low compared to other stages, and because complete protection at this stage has benefits for both the vaccinated person (e.g., no disease) and for elimination efforts (e.g., no transmission potential).

Antibodies (Ab) and cytotoxic T lymphocytes (CTL) contribute to sterile protection at the pre-erythrocytic stage by blocking invasion (Ab, Keitany et al.) and killing infected hepatocytes (CTL, Doolan and Hoffman), respectively. CTL that kill infected hepatocytes before release of viable merozoites (Doolan and Hoffman) are important for complete protection. However, thus far, durable sterile protection has only been achieved by repeated immunization with live attenuated sporozoites that arrest in the liver. Importantly, these sporozoite vaccines must be manufactured in mosquitoes and delivered by multiple intravenous (IV) injections.

Pre-clinical work showed that repeated sporozoite immunization resulted in stepwise reductions in liver burden at the time of vaccination dosing. This reduced infectivity of sequential vaccine booster doses was accompanied by reduced boosting against multiple T cell target antigens. Notably, clinical trials have shown a lack of CTL boosting for many antigens after the first sporozoite immunization (e.g., Mordmuller et al., 2017).

Antigen-specific responses have been minimally studied in humans since the outbred nature of human MHC makes epitope prediction difficult. Instead, inbred mice are valuable for understanding the expansion and contraction of specific T cell populations in responses to antigen presentation. Across both BALB/c (Murphy et al.) and C57BL/6 (Billman et al.) backgrounds, the collective data now show that antigen-specific CD8+ T cell responses to PyL3, PbS20₃₁₈₋₃₂₆, PbGAP50₄₁₋₄₈, PbF4, and PbNCY contract despite repeated immunization whereas responses to protective TRAP and CSP antigens remain stable or even expand. Responses that contract do not exclusively target late liver stage antigens. These data show that repeated sporozoite immunization results in a progressively more protective total immune response accompanied by a gradual ‘debulking’ of subsequent vaccine doses. This debulking explains the lack of secondary expansion of T cells targeting proteins like L3, F4 and NCY and possibly also S20. Debulking is at least partly due to antibodies directed against homologous parasites at the vaccination time point since circumventing such antibodies with heterologous sporozoites increased liver burden upon secondary vaccination (Billman et al.). These model antigens are likely emblematic of an entire class of antigens that fail to re-expand due to debulking of the secondary and later immunization doses by the immunity achieved by primary immunization. In summary, each successive booster dose of attenuated sporozoites is less and less effective, due in large part to transmission blocking antibodies induced by earlier sporozoites doses.

T cells residing in the liver are known as liver resident memory T cells. It is generally thought that when effector T cells encounter antigen in non-lymphoid tissues, a subset undergo transcriptional changes and differentially express cell-surface markers that restrict migration to the local milieu thus informing such T cells to remain as Trm. In the liver, Trm seeding can occur without overt inflammation (i.e. by antigen alone in the absence of an infectious agent). In the past year, CD8⁺ Trm cells were identified in pre-clinical Plasmodium studies as key components of pre-erythrocytic protection (Fernandez-Ruiz et al.).

Repeated immunizations with attenuated sporozoites are known to induce CTLs that establish liver Trm (Fernandez-Ruiz et al.) and that vaccine-induced cells can directly kill parasite-infected hepatocytes (Doolan and Hoffman; White et al.).

In humans, samples can only be obtained from peripheral blood. At the conclusion of vaccination, total sporozoite-specific cytokine-producing CD8+ T cells are more abundant in persons who go on to be protected against challenge as compared to those who are not protected (Seder et al.). However, the total percentage of parasite-specific cells in the periphery is extremely low (<0.5% of CD8s). Moreover, these T cell frequencies are lower than after the first sporozoite vaccination such that there is not apparent boosting of peripherally-measured T cell responses beyond the peak observed after the first dose of vaccine (Ishizuka et al.; Lyke et al.). Nonetheless, sporozoite-vaccinated human subjects can be protected at more than one-year post-vaccination at a time when parasite-specific antibody titers are low and non-protective (Ishizuka et al.). Thus, human data supports the idea that although antibodies may play a role in short term protection, liver resident memory T cells (Trm) are essential for long-term sterile protection (Ishizuka et al.). Although Trm cannot be measured directly in humans, they have been measured in mice and non-human primate livers. Parasite-specific, cytokine-producing CD8⁺ T cells in the livers of vaccinated animals were >10-100 times more abundant than the same cells in peripheral blood (Ishizuka et al.). Accordingly, the correlate of immunity in humans may be in the liver, an unmeasurable compartment.

Dendritic cell (DC) priming induced CD8⁺ T cells that could later be trapped in the liver using viral vectors that induced liver inflammation and parasite-specific antigen expression by hepatocytes (Fernandez-Ruiz et al.). These trapped cells form liver Trm, which were required for protection against malaria sporozoite challenge. Tissue resident memory T cells are increasingly appreciated in a wide array of organs including the lung (Strutt et al.), liver (Fernandez-Ruiz et al.; Tse et al.), central nervous system (Pavelko et al.), gastrointestinal tract (Kiniry et al.) and genital tract (Tan et al.).

Thus, vaccines capable of efficiently inducing liver Trm CTL for malaria would be highly desirable. A completely protective vaccine is urgently needed to reduce the burden of disease and to accelerate progress toward elimination of infections. However, progress toward a vaccine has been difficult and as such, it is of importance to develop alternative methods for vaccinating subjects against malaria.

### SUMMARY

In some embodiments, malaria vaccine compositions, associated regimens, associated methods, associated systems, and associated compositions. In particular, methods of method of vaccinating a mammal by administering a first composition comprising an antigenic subunit component to the mammal and administering a second composition comprising a liver-specific antigenic component to the mammal, are provided. The first and second compositions are not administered concurrently and wherein a number of resident memory T cells in the liver are increased following administration of the first and/or second compositions.

In certain embodiments, the antigenic subunit component of (i) is selected from the group consisting of (a) a wild-type or an attenuated Plasmodium parasite, (b) a deoxyribonucleic acid (DNA) polynucleotide, (c) a ribonucleic acid (RNA) polynucleotide, (d) a protein or a polypeptide, (e) a virally-vectored antigen, (f) a virus-like particle delivered antigen, (g) a fragment of any of (b), (c), (d), (e), or (f), (h) a subunit of (e) or (f), and, (i) a combination of any of (a), (b), (c), (d), (e), (f), (g), or (h). In certain embodiments, the liver-specific antigenic component of (ii) is selected from the group consisting of: (a) a wild-type or an attenuated Plasmodium parasite, (b) a deoxyribonucleic acid (DNA) polynucleotide, (c) a ribonucleic acid (RNA) polynucleotide, (d) a protein or a polypeptide, (e) a virally-vectored antigen, (f) a virus-like particle delivered antigen, (g) a fragment of any of (b), (c), (d), (e), or (f), (h) a subunit of (e) or (f), and, (i) a combination of any of (a), (b), (c), (d), (e), (f), (g), or (h). In some embodiments, the antigenic subunit component of (i) and/or the liver-specific antigenic component of (ii) is the DNA polynucleotide of (b) or the RNA polynucleotide of (c), and encodes a polypeptide comprising a circumsporozoite (CSP) fragment and/or another Plasmodium protein. In other embodiments, the antigenic subunit component of (i) and/or the liver-specific antigenic component of (ii) is the DNA polynucleotide of (b) or the RNA polynucleotide of (c), and encodes a polypeptide comprising a protein from another liver-tropic pathogen such as hepatitis C virus. In further embodiments, the antigenic subunit component of (i) and/or the liver-specific antigenic component of (ii) is the protein or polypeptide of (d) and/or the virus-like particle delivered antigen of (f) and comprises a CSP fragment and/or another Plasmodium protein. In still further embodiments, the antigenic subunit component of (i) and/or the liver-specific antigenic component of (ii) is the protein or polypeptide of (d) and/or the virus-like particle delivered antigen of (f) and comprises a protein from another liver-tropic pathogen such as hepatitis C virus. in some embodiments, the DNA polynucleotide of the first composition is the same as the DNA polynucleotide of the second composition. The polypeptide can include a tag, such as a ubiquitin tag. In addition, the first composition and/or second composition can be administered to the mammal with an adjuvant, such as an E. coli heat-labile toxin-encoding plasmid.

In some embodiments, the sporozoite component comprises one or more sporozoites selected from one or more Plasmodium species, one or more recombinant Plasmodium species or strains, one or more sporozoite strains, or a combination thereof. In some of these embodiments, one or more of the sporozoites are attenuated.

In certain embodiments, an antibody response to the first composition and/or the second composition is not induced in the mammal following administration of the first composition and/or the second composition. In other embodiments, an antibody response to the first composition and/or the second composition is induced in the mammal following administration of the first composition and/or the second composition. Following administration, the first composition primes CD8+ T cells. In some embodiments, a first number of resident memory CD8+ T (liver Trm) cells in the mammal's liver increases to a second number of Trm cells of following administration of the second composition. The first composition can be administered at least one day, at least two days; at least three days, at least four days, at least five days, at least six days, at least seven days, at least ten days, at least two weeks, at least three week, at least four weeks, at least six weeks, or at least eight weeks before the second composition is administered.

In some embodiments, the methods of vaccinating a mammal comprise the steps of (i) administering a first composition comprising an antigenic subunit component to the mammal, and (ii) administering a second composition comprising a wild-type or an attenuated Plasmodium parasite to the mammal. In these embodiments, the first and second compositions are not administered concurrently and wherein a number of resident memory T cells in the liver are increased following administration of the first and second compositions. In other embodiments, the methods of vaccinating a mammal comprise the steps of (i) administering a first composition comprising an antigenic subunit component to the mammal, and (ii) administering a second composition comprising a virally-vectored antigen to the mammal. In these embodiments, the first and second compositions are not administered concurrently and wherein a number of resident memory T cells in the liver are increased following administration of the first and second compositions. In certain embodiments, the antigenic subunit component of (i) is selected from the group consisting of, (a) a deoxyribonucleic acid (DNA) polynucleotide, (b) a ribonucleic acid (RNA) polynucleotide, (c) a protein or a polypeptide, (d) a virally-vectored antigen, (e) a virus-like particle delivered antigen, (f) a fragment of any of (b), (c), (d), or (e), (g) a subunit of (e) or (f), and, (h) a combination of any of (a), (b), (c), (d), (e), (f), or (g).

In some embodiments, the antigenic subunit component is the DNA polynucleotide of (a) or the RNA polynucleotide of (b), and encodes a polypeptide comprising a CSP fragment and/or another Plasmodium protein, or the antigenic subunit component is the DNA polynucleotide of (a) or the RNA polynucleotide of (b), and encodes a polypeptide comprising a protein from another liver-tropic pathogen such as hepatitis C virus. In other embodiments, the antigenic subunit component is the protein or polypeptide of (c) and/or the virus-like particle delivered antigen of (e), and comprises a CSP fragment and/or another Plasmodium protein, or the antigenic subunit component is the protein or polypeptide of (c) and/or the virus-like particle delivered antigen of (e), and comprises a protein from another liver-tropic pathogen such as hepatitis C virus. The polypeptide can include a tag, such as a ubiquitin tag. In addition, the first composition and/or second composition can be administered to the mammal with an adjuvant, such as an E. coli heat-labile toxin-encoding plasmid.

In certain embodiments, the sporozoite component comprises one or more sporozoites selected from one or more Plasmodium species, one or more recombinant Plasmodium species or strains, one or more sporozoite strains, or a combination thereof. One or more of the sporozoites can be attenuated.

In certain embodiments, an antibody response to the first composition and/or the second composition is not induced in the mammal following administration of the first composition and/or the second composition. In other embodiments, an antibody response to the first composition and/or the second composition is induced in the mammal following administration of the first composition and/or the second composition. Following administration, the first composition primes CD8+ T cells. In some embodiments, a first number of resident memory CD8+ T (liver Trm) cells in the mammal's liver increases to a second number of Trm cells of following administration of the second composition. The first composition can be administered at least one day, at least two days; at least three days, at least four days, at least five days, at least six days, at least seven days, at least ten days, at least two weeks, at least three week, at least four weeks, at least six weeks, or at least eight weeks before the second composition is administered.

In some embodiments, the methods of vaccinating a mammal comprise the steps of (i) administering a first composition comprising an antigenic subunit component including at least one deoxyribonucleic acid (DNA) polynucleotide to the mammal, and (ii) administering a second composition comprising a wild-type or attenuated plasmodium parasite to the mammal. In further embodiments, the methods of vaccinating a mammal comprise the steps of (i) administering a first composition comprising an antigenic subunit component including at least one ribonucleic acid (RNA) polynucleotide to the mammal, and (ii) administering a second composition comprising a wild-type or attenuated plasmodium parasite to the mammal. In still further embodiments, the methods of vaccinating a mammal comprise the steps of (i) administering a first composition comprising an antigenic subunit component including at least one deoxyribonucleic acid (DNA) polynucleotide to the mammal, and (ii) administering a second composition comprising a virally-vectored antigen to the mammal. In other embodiments, the methods of vaccinating a mammal comprise the steps of (i) administering a first composition comprising an antigenic subunit component including at least one ribonucleic acid (RNA) polynucleotide to the mammal, and (ii) administering a second composition comprising a virally-vectored antigen to the mammal. In any of the above embodiments, the first and second compositions are not administered concurrently and wherein a number of resident memory T cells in the liver are increased following administration of the first and second compositions.

In certain embodiments, the DNA polynucleotide encodes a polypeptide comprising a CSP fragment and/or another Plasmodium protein, or the RNA polynucleotide encodes a polypeptide comprising a CSP fragment and/or another Plasmodium protein. In certain embodiments, the DNA polynucleotide encodes a polypeptide comprising a protein from another liver-tropic pathogen such as hepatitis C virus, or the RNA polynucleotide encodes a protein from another liver-tropic pathogen such as hepatitis C virus. In certain embodiments, the wild-type or attenuated Plasmodium parasite is a CSP fragment and/or another Plasmodium protein, or the wild-type or attenuated Plasmodium parasite is a protein from another liver-tropic pathogen such as hepatitis C virus. In certain embodiments, the virally-vectored antigen is a CSP fragment and/or another Plasmodium protein, or the virally-vectored antigen is a protein from another liver-tropic pathogen such as hepatitis C virus.

The polypeptide can include a tag, such as a ubiquitin tag. In addition, the first composition and/or second composition can be administered to the mammal with an adjuvant, such as an E. coli heat-labile toxin-encoding plasmid.

In certain embodiments, the sporozoite component comprises one or more sporozoites selected from one or more Plasmodium species, one or more recombinant Plasmodium species or strains, one or more sporozoite strains, or a combination thereof. One or more of the sporozoites can be attenuated.

In certain embodiments, an antibody response to the first composition and/or the second composition is not induced in the mammal following administration of the first composition and/or the second composition. In other embodiments, an antibody response to the first composition and/or the second composition is induced in the mammal following administration of the first composition and/or the second composition. Following administration, the first composition primes CD8+ T cells. In some embodiments, a first number of resident memory CD8+ T (liver Trm) cells in the mammal's liver increases to a second number of Trm cells of following administration of the second composition. The first composition can be administered at least one day, at least two days; at least three days, at least four days, at least five days, at least six days, at least seven days, at least ten days, at least two weeks, at least three week, at least four weeks, at least six weeks, or at least eight weeks before the second composition is administered.

In some embodiments, a malarial vaccination regimen comprises (i) a first composition comprising an antigenic subunit component, and (ii) a second composition comprising a liver-specific antigenic component. In these embodiments, the antigenic subunit component of (i) is selected from the group consisting of, (a) a wild-type or an attenuated Plasmodium parasite, (b) a deoxyribonucleic acid (DNA) polynucleotide, (c) a ribonucleic acid (RNA) polynucleotide, (d) a protein or a polypeptide, (e) a virally-vectored antigen, (f) a virus-like particle delivered antigen, (g) a fragment of any of (b), (c), (d), (e), or (f), (h) a subunit of (e) or (f), and, (i) a combination of any of (a), (b), (c), (d), (e), (f), (g), or (h). Also in the embodiments, the liver-specific antigenic component of (ii) is selected from the group consisting of, (a) a wild-type or an attenuated Plasmodium parasite, (b) a deoxyribonucleic acid (DNA) polynucleotide, (c) a ribonucleic acid (RNA) polynucleotide, (d) a protein or a polypeptide, (e) a virally-vectored antigen, (f) a virus-like particle delivered antigen, (g) a fragment of any of (b), (c), (d), (e), or (f), (h) a subunit of (e) or (f), and, (i) a combination of any of (a), (b), (c), (d), (e), (f), (g), or (h).

In certain embodiments, the antigenic subunit component of (i) and/or the liver-specific antigenic component of (ii) is the DNA polynucleotide of (b) or the RNA polynucleotide of (c), and encodes a polypeptide comprising a CSP fragment and/or another Plasmodium protein, or the antigenic subunit component of (i) and/or the liver-specific antigenic component of (ii) is the DNA polynucleotide of (b) or the RNA polynucleotide of (c), and encodes a polypeptide comprising a protein from another liver-tropic pathogen such as hepatitis C virus. In certain embodiments, the antigenic subunit component of (i) and/or the liver-specific antigenic component of (ii) is the protein or polypeptide of (d) and/or the virus-like particle delivered antigen of (f), and comprises a CSP fragment and/or another Plasmodium protein, or the antigenic subunit component of (i) and/or the liver-specific antigenic component of (ii) is the protein or polypeptide of (d) and/or the virus-like particle delivered antigen of (f), and comprises a protein from another liver-tropic pathogen such as hepatitis C virus.

The polypeptide can include a tag, such as a ubiquitin tag. In addition, the first composition and/or second composition can be administered to the mammal with an adjuvant, such as an E. coli heat-labile toxin-encoding plasmid.

In certain embodiments, the sporozoite component comprises one or more sporozoites selected from one or more Plasmodium species, one or more recombinant Plasmodium species or strains, one or more sporozoite strains, or a combination thereof. One or more of the sporozoites can be attenuated.

In certain embodiments, an antibody response to the first composition and/or the second composition is not induced in the mammal following administration of the first composition and/or the second composition. In other embodiments, an antibody response to the first composition and/or the second composition is induced in the mammal following administration of the first composition and/or the second composition. Following administration, the first composition primes CD8+ T cells. In some embodiments, a first number of resident memory CD8+ T (liver Trm) cells in the mammal's liver increases to a second number of Trm cells of following administration of the second composition. The first composition can be administered at least one day, at least two days; at least three days, at least four days, at least five days, at least six days, at least seven days, at least ten days, at least two weeks, at least three weeks, at least four weeks, at least six weeks, or at least eight weeks before the second composition is administered.

In some embodiments, a malarial vaccination regimen comprises (a) a first composition comprising a first DNA component, and (b) a second composition comprising a wild-type or attenuated Plasmodium parasite. In other embodiments, a malarial vaccination regimen comprises (a) a first composition comprising a first RNA component, and (b) a second composition comprising a wild-type or attenuated Plasmodium parasite. In still other embodiments, a malarial vaccination regimen comprises (a) a first composition comprising a protein or polypeptide component, and (b) a second composition comprising a wild-type or attenuated Plasmodium parasite. In further embodiments, a malarial vaccination regimen comprises (a) a first composition comprising a virally-vectored antigen, and (b) a second composition comprising a wild-type or attenuated Plasmodium parasite. In still further embodiments, a malarial vaccination regimen comprises (a) a first composition comprising a virus-like particle delivered antigen, and (b) a second composition comprising a wild-type or attenuated Plasmodium parasite. In yet other embodiments, a malarial vaccination regimen comprises (a) a first composition comprising a virally-vectored antigen, and (b) a second composition comprising a wild-type or attenuated Plasmodium parasite. In yet further embodiments, a malarial vaccination regimen comprises (a) a first composition comprising a first DNA component, and (b) a second composition comprising a virally-vectored antigen. In yet additional embodiments, a malarial vaccination regimen comprises (a) a first composition comprising a first RNA component, and (b) a second composition comprising a virally-vectored antigen. In still yet further embodiments, a malarial vaccination regimen comprises (a) a first composition comprising a protein or polypeptide component, and (b) a second composition comprising a virally-vectored antigen. In still yet other embodiments, a malarial vaccination regimen comprises (a) a first composition comprising a virally-vectored antigen, and (b) a second composition comprising a virally-vectored antigen. In still yet additional embodiments, a malarial vaccination regimen comprises (a) a first composition comprising a virus-like particle delivered antigen, and (b) a second composition comprising a virally-vectored antigen. In still yet further embodiments, a malarial vaccination regimen comprises (a) a first composition comprising a virally-vectored antigen, and (b) a second composition comprising a virally-vectored antigen.

In certain embodiments, the first composition and/or the second composition encodes or is a polypeptide comprising a CSP fragment and/or another Plasmodium protein, or the first composition and/or the second composition encodes or is a polypeptide comprising a protein from another liver-tropic pathogen such as hepatitis C virus.

The polypeptide can include a tag, such as a ubiquitin tag. In addition, the first composition and/or second composition can be administered to the mammal with an adjuvant, such as an E. coli heat-labile toxin-encoding plasmid.

In certain embodiments, the sporozoite component comprises one or more sporozoites selected from one or more Plasmodium species, one or more recombinant Plasmodium species or strains, one or more sporozoite strains, or a combination thereof. One or more of the sporozoites can be attenuated.

In certain embodiments, an antibody response to the first composition and/or the second composition is not induced in the mammal following administration of the first composition and/or the second composition. In other embodiments, an antibody response to the first composition and/or the second composition is induced in the mammal following administration of the first composition and/or the second composition. Following administration, the first composition primes CD8+ T cells. In some embodiments, a first number of resident memory CD8+ T (liver Trm) cells in the mammal's liver increases to a second number of Trm cells of following administration of the second composition. The first composition can be administered at least one day, at least two days; at least three days, at least four days, at least five days, at least six days, at least seven days, at least ten days, at least two weeks, at least three weeks, at least four weeks, at least six weeks, or at least eight weeks before the second composition is administered.

In some embodiments, the malarial vaccination regimen includes two or more of the malarial vaccination regimens of the present technology. In some embodiments, the first composition targets multiple antigens and/or the second composition targets multiple antigens.

In some embodiments, a method for increasing a number of liver Trms in a mammal, comprises administering to the mammal one or more of the malarial vaccination regimens of the present technology. In certain embodiments, the second composition of the one or more malarial vaccination regimens further comprises a viral vector encoding a malarial antigen. In other embodiments, the viral vector is an adeno-associated viral vector, a yellow fever viral vector, an adenoviral vector, a modified vaccinia virus ankara, or a combination thereof.

In some embodiments, a method for tissue-specific vaccination in mammals, comprises the steps of (a) administering to the mammal a first composition comprising an antigenic subunit component, and (b) administering to the mammal a second composition comprising a tissue-specific component of an infectious organism. In these embodiments, the first and second compositions are not administered concurrently, and a number of Trm cells in the subject's specific tissue are increased.

In certain embodiments, the infectious organism is malaria and/or the tissue-specific component is a sporozoite. The tissue-specific vaccination can be specific for the mammal's liver tissue. In further embodiments, the antigenic subunit component is selected from the group consisting of (a) a wild-type or an attenuated Plasmodium parasite, (b) a deoxyribonucleic acid (DNA) polynucleotide, (c) a ribonucleic acid (RNA) polynucleotide, (d) a protein or a polypeptide, (e) a virally-vectored antigen, (f) a virus-like particle delivered antigen, (g) a fragment of any of (b), (c), (d), (e), or (f), (h) a subunit of (e) or (f), and, (i) a combination of any of (a), (b), (c), (d), (e), (f), (g), or (h). In some embodiments, the second composition further comprises the antigenic subunit component.

### BRIEF DESCRIPTION OF THE DRAWINGS

Many aspects of the present technology can be better understood with reference to the following drawings. Emphasis is placed on illustrating clearly the principles of the present technology.

FIGS. 1A and 1B illustrate a lack of protection using DNA-only circumsporozoite (CSP) vaccine. n=5 control mice and n=9 CSP-vaccinated mice. (A) CSP-specific CD8+ T cells as a percentage of all CD8⁺ T cells in peripheral blood four days prior to challenge are shown. (B) Sterile protection in mice was assessed by peripheral blood smears.

FIGS. 2A-2C illustrate an acute challenge model. n=4/group. (A) Peak CSP-specific cytotoxic T-lymphocytes (CTL) frequency six days post-DNA booster is shown. (B) High dose (5×10⁴ luc-expressing and low dose (5×10² wild-type) are depicted. Liver burden was assessed 44 hours post-challenge by an in vitro imaging system (IVIS). (C) A sporozoite challenge performed five days after the final deoxyribonucleic acid (DNA) booster for the indicated antigen or control vector. Sterile protection was assessed by peripheral blood smears.

FIG. 3 illustrates the flow cytometry gating strategy used to identify CSP-specific liver Trm.

FIGS. 4A-4C illustrate that DNA prime-boosting with hydrodynamic transfection (HDT) delivery of antigen at the boost results in high frequency liver Trm, and is associated with rapid clearance of antigen-expressing hepatocytes. (A) Area under the curve (AUC) for days 0-7 for total flux of HDT csp-t2a-luc was measured for naïve or CSP-immunized mice divided by mean AUC for similarly immunized mice given luc-only HDT. (B) Example IVIS images for mice in the prime→boost groups after receipt of luc HDT (left) or csp-luc HDT (right). (C) Liver Trm data on mice in groups illustrated by the first two bars in (A) one month after completion of HDT using csp-luc or luc only.

FIGS. 5A-5C illustrate results from a DNA prime→DNA-plus-sporozoite “Trap” study in CSP-specific Trm that exceed the frequency usually observed for sporozoite hyperimmunized mice. Priming on day 0→boosting on day 28→optional boost on day 33 as indicated by X-axis text and arrows. Mice were sacrificed and evaluated on day 56. *p<0.05; **p<0.01; ***p<0.0001; ns, not significant. (A) Liver-specific CSP-specific CD8⁺ Trm. (B) CSP-specific effector T cells in the liver. (C) CSP-specific effector T cells in spleen in BALB/c mice immunized as indicated.

FIG. 6 shows that complete protection was achieved following a DNA→(DNA plus sporozoite) prime and trap vaccination. Mice in the listed vaccinations groups were challenged with 500 wild-type P. yoelii sporozoites intravenously (IV) and monitored by blood smears from Day 4-10 post-challenge. n=5 mice/group except n=3/group for the RAS only group.

FIGS. 7A and 7B illustrate models of CSP-only and sporozoite-only vaccines and development of liver CD8⁺ Trm. Legend defines icons. (A) DNA-only vaccination achieves high effector CD8+ T cell frequencies in the spleen but minimal liver Trm. (B) Triple immunization with attenuated sporozoites induces protective high frequency liver Trm, which are also induced by DNA→(DNA+sporozoites).

### DETAILED DESCRIPTION

### Overview

The present technology is directed generally to vaccine compositions, delivery regimens, methods of administration, associated methods, and associated systems, for treating an infections organism, such as malaria. Without intending to be bound by any particular theory, it is thought that antibodies generated by the subject against one or more malarial vaccine components Antibodies can work against efficient boosting of liver Trm CTLs. The present technology differs from prior malarial vaccine compositions, associated regimens, and associated methods by priming the subject's CTLs using a single trapping dose of sporozoites or, in some embodiments, the present technology does not include doses of any sporozoites.

Unlike conventional malaria vaccine compositions, malaria vaccine compositions of the present technology, delivery regimens for these malaria vaccine compositions, methods of administering malaria vaccine compositions, associated methods, and associated systems which include nucleic acid (e.g., DNA or RNA) components, are each administered to the subject at least once and at different times. In some embodiments, the DNA and/or RNA components can have different formulations. As such, the present technology utilizes a modified prime-and-trap approach. In certain embodiments, the present modified prime-and-trap approach is directed to a regimen that involves two compositions, the priming composition and the boosting/trapping composition (e.g., the first composition and the second composition). The first composition includes a priming component (e.g., a first antigenic subunit component) able to elicit an immune response, such as a first nucleic acid molecule-based vaccine (i.e., a viral or plasmid vector-based DNA or RNA vaccine). The second composition includes (i) a second antigenic subunit component (e.g., a second DNA or RNA vaccine) to boost or bolster the effects of the priming component and (ii) a trapping component that directs (or traps) the first and/or second antigenic subunit component to a target tissue. In certain embodiments, the trapping component described herein is a liver-specific antigenic component and may also serve to boost or bolster the effects of the priming component.

In some embodiments, the first and second antigenic subunit components comprise first and second DNA and/or RNA vaccines, respectively. The DNA and/or RNA vaccines can include one or more DNA and/or RNA sequences, such as antigen-coding sequences. In some embodiments, the one or more DNA and/or RNA sequences are the same as one another, and, in other embodiments, the one or more DNA sequences are different from one another. For example, the first DNA and/or RNA vaccine may include a first group of multiple different priming DNA and/or RNA sequences administered in parallel (e.g., a group comprising two or more different sequences, three or more different sequences, four or more different sequences, four or more different sequences, five or more different sequences, six or more different sequences, seven or more different sequences, eight or more different sequences, nine or more different sequences, ten or more different sequences, twenty or more different sequences, thirty or more different sequences, forty or more different sequences, fifty or more different sequences, sixty or more different sequences, seventy or more different sequences, eighty or more different sequences, ninety or more different sequences, 100 or more different sequences, more than 100 different sequences, more than 1000 different sequences, a plurality of different sequences, and so on). Likewise, the second DNA and/or RNA vaccine may include a second group of multiple different boosting DNA and/or RNA sequences administered in parallel. The first group of sequences in the first DNA and/or RNA vaccine and the second group of sequences in the second DNA and/or RNA vaccine may be the same group of sequences, a different group of sequences, or a combination of some of the same sequences and some different sequences. In addition, the one or more antigen-coding sequences can include one or more adjacent DNA sequences that are the same or different from one another. For example, in some embodiments, a DNA and/or RNA sequence in the first DNA and/or RNA vaccine may have the same antigen-coding sequence as a DNA and/or RNA sequence in the second DNA and/or RNA vaccine but may differ with respect to a sequence adjacent to the antigen-coding sequence. For example, the adjacent promoter sequence may differ based on the target delivery method or target tissue (e.g., a DNA priming in the skin may require a CMV promoter, while a liver-specific boosting dose may require a different liver-specific promoter. Without intending to be bound by any particular theory, it is thought that the subject elicits a response to each of the antigen-coding sequences in the DNA vaccine. In some embodiments, the DNA vaccine can be included in the trapping component. In some embodiments, the second antigenic subunit component and the trapping component are administered at the same time (e.g., as the second composition).

In accordance with the embodiments described herein, malaria vaccine compositions and regimens that target the pre-erythrocytic stage use a trapping component that directs the effects of the first and/or second antigenic subunit component to the liver, such as the liver-specific component. For example, the trapping component may include one or more tissue-specific antigens of an infectious organism—specifically, one or more liver antigens of malaria. Such liver-specific antigens of malaria may include, but are not limited to, a wild-type or attenuated sporozoite or one or more sporozoite surface antigens or functional fragments thereof. In other examples, the trapping component may include a non-infectious liver-specific antigen or functional fragment or epitope thereof. In certain aspects of this embodiment, the second antigenic subunit component of the second composition is not necessary to impart partial or sterile immunity. In those aspects, the second composition includes the trapping component but does not include the second antigenic subunit component (e.g., the vaccine moiety used for the priming vaccination). In this embodiment, the second composition is administered between about 7 days to about 56 days, inclusive, after the first composition as described in detail below. For example, the second composition is administered about 28 days after the first composition as described in detail below

In another embodiment, the regimen involves three compositions, a first antigenic subunit composition (e.g., the first composition), a trapping composition (e.g., the second composition), and a second antigenic subunit composition (e.g., the third composition). The first antigenic subunit composition includes a priming component able to elicit an immune response, such as a first viral or plasmid vector-based DNA or RNA vaccine as described above. The trapping composition includes a trapping component, such as those described herein. The second antigenic subunit composition includes a second viral or plasmid vector-based DNA or RNA vaccine as described above. The second viral or plasmid vector-based DNA or RNA vaccine may be the same DNA or RNA vaccine used in the priming composition, or may be different, as described above. In this embodiment, second antigenic subunit composition and the trapping composition are administered at different times and as different compositions. For example, the second antigenic subunit composition is administered before the trapping composition is administered. More specifically, the trapping composition is administered between about 7 days to about 56 days, inclusive after the first antigenic subunit composition as described in detail below. The DNA or RNA second antigenic subunit composition is optional and is administered simultaneously with or in conjunction with the trapping composition, meaning that the second antigenic subunit composition may be administered at the same time, just before, or just after the trapping composition.

Without intending to be bound by any particular theory, it is thought that the present technology improves induction of liver Trm and reduces the number of vaccine doses needed to achieve sterile immunity compared to conventional malaria vaccine compositions and associated methods. As shown in the present application, DNA-only vaccines avoided antibody responses in the subject following administration, thereby allowing a single use of a sporozoite trapping dose. Accordingly, the present technology includes a heterologous prime-and-trap strategy for malaria vaccination that results in complete protection with as few as two doses of the malaria vaccine composition (e.g., a first dose of the first composition and a first dose of the second composition). In some embodiments, the heterologous prime-and-trap strategy primes antigen-specific CD8⁺ T cells in a subject following administration of a first composition of the malaria vaccine composition (e.g., a DNA or RNA vaccine) without inducing concurrent antibody responses and boosts the subject with a second composition of the malaria vaccine composition (e.g., vaccine comprising the same DNA as the first composition and attenuated sporozoites). Without intending to be bound by any particular theory, it is thought that following administration, the attenuated sporozoites home to the liver, express liver antigens, and attract previously primed and expanding populations of CD8⁺ T cells to the liver, where they become Trm. In some embodiments and as described in greater detail herein, delivery regimens for malaria vaccine compositions and methods of administering malaria vaccine compositions include the gene gun approach.

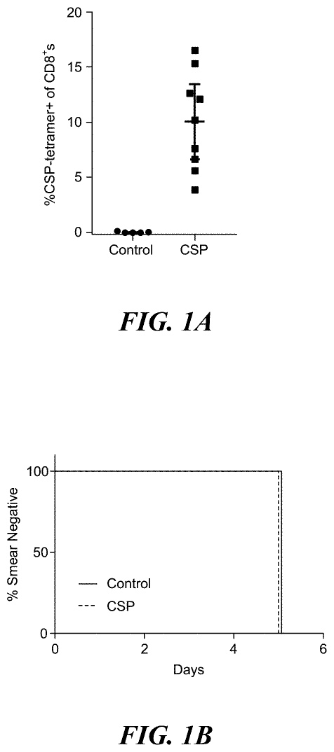

Specific details of some embodiments of the present technology are described below with reference to malaria vaccine compositions, malaria vaccination regimens, and/or methods for administering one or more of the malaria vaccine compositions and/or malaria vaccination regimens to a subject in need, and associated systems for determining certain aspects of the methods. Some embodiments can have configurations, components and/or procedures different than those which are described herein, and other embodiments may eliminate particular components or procedures. The present disclosure may include some embodiments with additional elements, and/or may include some embodiments without several of the features shown and described below with reference to FIGS. 1A-7.

The term “tissue specific antigen of an infectious organism” refers to an antigen of an infectious organism that can elicit an immune response in a target tissue. As it relates to the malaria vaccines, compositions, regimens, methods of administration, associated methods, and associated systems for treating pre-erythrocytic malaria, described herein, the tissue-specific antigen is a protective antigen that is expressed in the liver that may be a liver-specific antigen (i.e., expressed only in the subject's liver or substantially only in the subject's liver), an antigen that is expressed in the subject's liver but are expressed outside of the subject's liver as well, or an antigen that is expressed outside of the subject's liver but is found in the liver. A non-limiting example of a tissue specific antigen of an infectious organism includes a sporozoite antigen that is expressed and/or present in the liver stage. The infectious organism is Plasmodium. This term is used interchangeably throughout the application with the terms “a wild-type or attenuated sporozoite” and “a wild-type or an attenuated Plasmodium parasite”.

As used herein the term “subject” or “subjects” refers to an animal, a mammal, a non-human primate, and/or a human in both the singular and the plural form (e.g., more than one).

Unless otherwise stated, the terms “about” and “approximately” refer to values within 10% of a stated value.

### Malaria Vaccine Compositions

The malaria vaccine compositions (e.g., malaria vaccines) described herein include a first composition, including first (or priming) antigenic component and a second composition, including a second (or boosting trapping) antigenic component. The second antigenic component may include one or more boosting components, for example, the second composition may include a second antigenic subunit component, a trapping component, or both. The trapping component may act as a boosting antigenic component. Either the priming antigenic component and/or the boosting/trapping antigenic component is selected from the group consisting of a wild-type or an attenuated Plasmodium parasite, a deoxyribonucleic acid (DNA) polynucleotide, a ribonucleic acid (RNA) polynucleotide, a protein or a polypeptide, a virally-vectored antigen, a virus-like particle delivered antigen, a fragment thereof, a subunit thereof, and/or a combination thereof. In some embodiments, the priming antigenic component includes one or more of an antigenic subunit component, a DNA component, an RNA component, a protein or polypeptide component, a virally-vectored antigen, a virus-like particle delivered antigen, and/or a combination thereof. The boosting antigenic component includes, but is not limited to, a liver-specific antigenic component, such as a wild-type or attenuated Plasmodium parasite, a virally-vectored antigen, and/or a combination thereof. In some embodiments, a component of the first composition can be the same as a component of the second composition. For example, both the first composition and the second composition can include a DNA polynucleotide. In addition, the DNA polynucleotide of the first composition can have the same nucleotide sequence as the DNA polynucleotide of the second composition.

In some embodiments, the second composition of the malaria vaccine compositions further includes a viral vector encoding a malarial antigen. For example, the viral vector is an adeno-associated viral vector, a yellow fever viral vector, an adenoviral vector, a modified vaccinia virus ankara, or a combination thereof.

In some embodiments, the malaria vaccine compositions are tissue-specific vaccines. As described herein, a tissue-specific vaccine refers to a vaccine that targets and/or operates in a tissue-specific manner, thereby mounting an immune response by cells residing in a specific tissue. For example, the vaccines described herein can provide pre-erythrocytic protection by targeting Trm cells to reside in the liver. Thus, according to certain embodiments, tissue-specific vaccines include vaccines specific for the subject's liver tissue.

Malaria vaccine compositions of the present technology target one or more antigens. For example, the antigenic subunit component and/or the liver-specific antigenic component can target one antigen, two antigens, three antigens, four antigens, five antigens, six antigens, eight antigens, nine antigens, ten antigens, 15 antigens, or 20 antigens. In some embodiments, malaria vaccine compositions target a greater number of antigens when intended for use in humans compared to those intended for use in other non-human subjects due to the plurality of human major histocompatibility complex (MHC) genes in humans compared to mice.

Without intending to be bound by any particular theory, it is thought that the one or more antigens targeted by the malaria vaccine compositions of the present technology can active one or more CD8⁺ T cells which detect and eliminate one or more liver cells infected with the one or more antigens (Longley et al.). In some embodiments, the antigen of the antigenic subunit component and/or the liver-specific antigenic component is the DNA polynucleotide or the RNA polynucleotide, and encodes a polypeptide comprising a circumsporozoite (CSP) fragment and/or another Plasmodium protein. In other embodiments, the antigenic subunit component and/or the liver-specific antigenic component is the DNA polynucleotide or the RNA polynucleotide, and encodes a polypeptide comprising a protein from another liver-tropic pathogen such as hepatitis C virus. In further embodiments, the antigenic subunit component and/or the liver-specific antigenic component is the protein or polypeptide and/or the virus-like particle delivered antigen, and comprises the CSP fragment and/or other Plasmodium protein(s). In still further embodiments, the antigenic subunit component and/or the liver-specific antigenic component is the protein or polypeptide and/or the virus-like particle delivered antigen, and comprises a protein from another liver-tropic pathogen such as hepatitis C virus.

In some embodiments, one or more antigens targeted by the malaria vaccine compositions of the present technology include one or more non-CSP antigens which may mediate protection without inducing CSP-specific immunity (Gruner et al.; Kumar et al.; Mauduit et al.). In addition to CSP, the non-CSP antigens include more than about 2000 pre-erythrocytic proteins that may or may not be targeted by pre-erythrocytic humoral and CTL responses. For example, of these more than about 2000 additional antigens, specific antigens that the antigenic subunit component of the malaria vaccine compositions can target include, but are not limited to, thrombospondin-related adhesive protein (TRAP/SSP2) (Pearson et al and Longley et al.), PfLSA1, PfAMA1, CeITOS (Mishra et al.), PfLSA3 (Sauzet et al.), the ortholog of PBANKA_071450 (Lau et al.), the ortholog of PY03470 (Cherif et al.), the ortholog of PY06414/TMP21 (Chen et al.), the ortholog of Py03011 (Limbach et al.), the ortholog of Py03424 (Limbach et al.), the ortholog of Py03661 (Limbach et al.), the ortholog of PY01316 (Haddad et al.), the ortholog of Py01157 (Zhang et al.), and/or a combination thereof.

Malaria vaccine compositions of the present disclosure may further include one or more polypeptides having one or more tags, such as a tagged-polypeptide. In some embodiments, the tag is a ubiquitin tag however, in other embodiments, one or more polypeptides can include a different tag or additional tags, such as a poly-histidine (e.g., 6X-HIS), chitin binding protein (CBP), maltose binding protein (MBP), streptavidin (SA), glutathione-S-transferase (GST), calmodulin-tag, E-tag, FLAG-tag, hemagglutinin tag (HA), c-myc tag, LC3 tag and any other tag suitable for conjugation to a polypeptide and useful with the present disclosure. In some embodiments, malaria vaccine compositions of the present disclosure may optionally include an adjuvant or adjuvant-encoding plasmids. For example, the first composition and/or second composition of the malaria vaccine compositions can be administered to a subject in need thereof (e.g., a mammal) with the adjuvant. In some embodiments, the adjuvant is an E. coli heat-labile toxin-encoding plasmid.

In some embodiments, the wild-type or attenuated Plasmodium parasite is a wild-type or attenuated sporozoite from a species of malaria. For example, the second composition of the malaria vaccine composition includes one or more wild-type or attenuated sporozoites selected from one or more Plasmodium species, such as but not limited to, P. falciparum, P. vivax, P. ovale, and P. malariae, one or more recombinant Plasmodium species or strains, one or more sporozoite strains, or a combination thereof. The second composition of the malaria vaccine composition can include one or more sporozoites from one or more additional malaria species and/or sub-species or can include one or more sporozoites from one or more malaria species and/or sub-species instead of P. falciparum, P. vivax, P. ovale, and P. malariae. In some embodiments, one or more of the sporozoites are attenuated. Without intending to be bound by any particular theory, it is thought that a sporozoite may be a “trap” and induce trafficking of one or more cells (e.g., immune cells such as antigen presenting cells, T cells, or the like) to the subject's liver.

Unlike conventional malaria vaccines, malaria vaccine compositions of the present technology do not necessarily induce an antibody response in the subject against one or more components of the malaria vaccine itself following administration. In this way, and without intending to be bound by any particular theory, the present technology results in malaria vaccine compositions, malaria vaccine regimens, and malaria vaccine methods that are more efficacious when compared to conventional malaria vaccines. In some embodiments, the antibody response to the first composition and/or the second composition in the subject following administration of one or more of the malaria vaccine compositions is not induced in the mammal following administration of the first composition and/or the second composition. However, in other embodiments, the antibody response to the first composition and/or the second composition in the subject following administration of one or more of the malaria vaccine compositions is induced in the mammal following administration of the first composition and/or the second composition. In these embodiments, the subject's antibody response to the first composition and/or the second composition may be reduced compared to a subject who received a conventional malaria vaccine.

### Malaria Vaccination Regimens

Malaria vaccination regimens of the present technology include, but are not limited to, regimens including one or more of the malaria vaccine compositions (e.g., malaria vaccines) described herein. In some embodiments, a malarial vaccination regimen includes a first composition comprising a first antigenic subunit component and a second composition comprising a liver-specific antigenic component and, optionally, a second antigenic subunit component. In certain embodiments, first composition comprises a first antigenic subunit component, and the second composition comprises the liver-specific antigenic component (e.g., the trapping/boosting component). The first antigenic subunit component can be the same as the second antigenic subunit component or different. For example, the first antigenic subunit component can be a DNA polynucleotide(s) having the same sequence as the DNA polynucleotide(s) of the second antigenic subunit component. In these embodiments, the second antigenic subunit component and the liver-specific antigenic component are administered at the same time (e.g., as the second composition).

The malarial vaccination regimens of the present technology include a prime and trap approach where priming occurs following administration of the first composition and trapping occurs following administration of the second composition. Unlike conventional malaria vaccine regimens, malarial vaccination regimens of the present technology do not require, but can include, a DNA or RNA polynucleotide in the second composition. Accordingly, in some embodiments, the first composition is a priming composition and includes the DNA polynucleotide whereas the second composition is a trapping composition and includes one or more sporozoites.

In other embodiments of malaria vaccination regimens of the present technology, at least three compositions are administered to the subject in need thereof. In these embodiments, the malaria vaccination regimens include the priming composition (e.g., the first composition), the second antigenic subunit composition (e.g., the second composition), and the trapping composition (e.g., the third composition). Unlike malaria vaccine regimens having two compositions, the second antigenic subunit composition and the trapping composition are administered at the same or different times in malaria vaccine regimens having three compositions. For example, the priming composition is administered to the subject before second antigenic subunit composition, which is administered before or at the same time as the trapping composition.

In addition, malarial vaccination regimens of the present technology can further include two or more malarial vaccine regimens described herein.

As explained above, the malaria vaccines useful with the malaria vaccine regimens of the present technology include, but are not limited to, a first composition, such as a priming antigenic component and a second composition, such as a second antigenic subunit component. Either the priming antigenic component and/or the second antigenic subunit component is selected from the group consisting of a wild-type or an attenuated Plasmodium parasite, a deoxyribonucleic acid (DNA) polynucleotide, a ribonucleic acid (RNA) polynucleotide, a protein or a polypeptide, a virally-vectored antigen, a virus-like particle delivered antigen, a fragment thereof, a subunit thereof, and/or a combination thereof. In some embodiments, the priming antigenic component includes one or more of an antigenic subunit component, a DNA component, an RNA component, a protein or polypeptide component, a virally-vectored antigen, a virus-like particle delivered antigen, and/or a combination thereof. The second antigenic subunit component includes, but is not limited to, a liver-specific antigenic component, such as a wild-type or attenuated Plasmodium parasite, a virally-vectored antigen, and/or a combination thereof.

In some embodiments, a malarial vaccination regimen of the present technology includes a first composition comprising a first DNA component, and a second composition comprising a wild-type or attenuated Plasmodium parasite. In some embodiments, a malarial vaccination regimen of the present technology includes a first composition comprising a first RNA component, and a second composition comprising a wild-type or attenuated Plasmodium parasite. In some embodiments, a malarial vaccination regimen of the present technology includes a first composition comprising a protein or polypeptide component, and a second composition comprising a wild-type or attenuated Plasmodium parasite. In some embodiments, a malarial vaccination regimen of the present technology includes a first composition comprising a virally-vectored antigen, and a second composition comprising a wild-type or attenuated Plasmodium parasite. In some embodiments, a malarial vaccination regimen of the present technology includes a first composition comprising a virus-like particle delivered antigen, and a second composition comprising a wild-type or attenuated Plasmodium parasite. In some embodiments, a malarial vaccination regimen of the present technology includes a first composition comprising a virally-vectored antigen, and a second composition comprising a wild-type or attenuated Plasmodium parasite.

In some embodiments, a malarial vaccination regimen of the present technology includes a first composition comprising a first DNA component, and a second composition comprising a virally-vectored antigen. In some embodiments, a malarial vaccination regimen of the present technology includes a first composition comprising a first RNA component, and a second composition comprising a virally-vectored antigen. In some embodiments, a malarial vaccination regimen of the present technology includes a first composition comprising a protein or polypeptide component, and a second composition comprising a virally-vectored antigen. In some embodiments, a malarial vaccination regimen of the present technology includes a first composition comprising a virally-vectored antigen, and a second composition comprising a virally-vectored antigen. In some embodiments, a malarial vaccination regimen of the present technology includes a first composition comprising a virus-like particle delivered antigen, and a second composition comprising a virally-vectored antigen. In some embodiments, a malarial vaccination regimen of the present technology includes a first composition comprising a virally-vectored antigen, and a second composition comprising a virally-vectored antigen.

Regardless of the malaria vaccine regimen described herein, the first composition is administered to the subject before the second composition. In some embodiments, in any of these malaria vaccine regimens, the first composition can be administered to the subject at least one day, at least two days; at least three days, at least four days, at least five days, at least six days, at least seven days, at least ten days, at least two weeks, at least three weeks, at least four weeks, at least six weeks, or at least eight weeks before the second composition is administered to the subject. For example, the first composition is administered to the subject on day 0 and the second composition is administered to the subject on day 28 (e.g., 28 days after administration of the first composition). A third composition is optionally administered to the subject on day 2 (e.g., 2 days after administration of the first composition) and, includes but is not limited to, a DNA polynucleotide and/or one or more sporozoites, such as those described herein.

### Methods of Vaccinating Subjects Using One or More Malaria Vaccines

Methods of vaccinating subjects with one or more of the malaria vaccine compositions and/or malaria vaccination regimens of the present technology include, methods of vaccinating subjects by administering the first composition and the second composition to the subject. In some embodiments, the first and second compositions are not administered concurrently. In other embodiments, the first and second compositions are administered concurrently

In some embodiments, methods of vaccinating a mammal include administering a first composition comprising an antigenic subunit component to the mammal and administering a second composition comprising a wild-type or an attenuated Plasmodium parasite to the mammal. In other embodiments, methods of vaccinating a mammal include administering a first composition comprising an antigenic subunit component to the mammal and administering a second composition comprising a virally-vectored antigen to the mammal. In further embodiments, methods of vaccinating a mammal include administering a first composition comprising an antigenic subunit component including at least one deoxyribonucleic acid (DNA) polynucleotide to the mammal and administering a second composition comprising a wild-type or attenuated plasmodium parasite to the mammal. In still further embodiments, methods of vaccinating a mammal include administering a first composition comprising an antigenic subunit component including at least one ribonucleic acid (RNA) polynucleotide to the mammal and administering a second composition comprising a wild-type or attenuated plasmodium parasite to the mammal. In additional embodiments, methods of vaccinating a mammal include administering a first composition comprising an antigenic subunit component including at least one deoxyribonucleic acid (DNA) polynucleotide to the mammal and administering a second composition comprising a virally-vectored antigen to the mammal. In yet additional embodiments, methods of vaccinating a mammal include administering a first composition comprising an antigenic subunit component including at least one ribonucleic acid (RNA) polynucleotide to the mammal and administering a second composition comprising a virally-vectored antigen to the mammal. In some embodiments, the first and second compositions are not administered concurrently. In other embodiments, the first and second compositions are administered concurrently.

In some embodiments, the methods of vaccinating subjects with one or more of the malaria vaccine compositions and/or malaria vaccination regimens of the present technology result in tissue-specific vaccination in the subject. In these embodiments, the tissue is the subject's liver, and/or the vaccination is against malaria, such as the plurality of species and sub-species of malaria described herein. For example, the methods for tissue-specific vaccination in a subject includes administering a first composition comprising an antigenic subunit component to the subject and administering a second composition comprising a tissue-specific component of an infectious organism to the subject.

Methods of vaccinating subjects with one or more of the malaria vaccine compositions and/or malaria vaccination regimens of the present technology also include methods for increasing a number of resident memory T cells (e.g., liver Trms) in a subject, comprising administering to the subject one or more of the malarial vaccination regimens described herein. Following administration of the one or more malaria vaccine compositions to the subject, a number of resident memory T cells in the subject's liver are increased. For example, the number of resident memory T cells in the subject's liver are increased following administration of the first and second compositions of the malaria vaccine compositions.

Malaria vaccine compositions can be administered to the subject using any of the malaria vaccine regimens described herein which can further include administration using a gene gun method, and/or other suitable techniques. The gene gun method is well-known in the art and are described in Fuller and Dean, the entireties of which are incorporated herein by reference. The gene gun method, and other methods described herein, can be combined with Highly Parallel Immunization (HPI) technology described in WIPO Patent Publication No 2017/024084 (PCTUS2016/045439) which is incorporated herein by reference in its entirety) which could result in the malaria vaccine compositions achieving a greater T cell repertoire without immune “skewing” compared to methods in the absence of HPI technology.

In some embodiments, the gene gun method includes combining the first composition and or the second composition with a plurality of gold beads having a diameter of about 1 μm. The gold beads serve as a scaffold for the DNA and/or RNA polynucleotide component(s) of the malaria vaccine compositions that can be present in the first composition and/or the second composition. Once combined with the plurality of gold beads, the scaffold (e.g., gold beads and polynucleotides) are delivered sub-dermally to the subject in need thereof using a pulse of helium gas. Amounts of gold beads, DNA and/or RNA polynucleotides, helium gas, delivery parameters such as pressure, and additional techniques associated with the gene gun technology are readily be determined. Unlike other methods of delivering polynucleotides to a subject (e.g., delivery of naked DNA), the gene gun method includes delivery of at least about 10-fold, about 100-fold, about 1000-fold, or about 10,000-fold less DNA to the subject in need thereof. Without intending to be bound by any particular theory, delivering the first composition and/or the second composition using the gene gun method is thought to activate one or more dendritic cells to induce an antigen-specific response in the subject to the plurality of expression signals encoded in the DNA and/or RNA polynucleotide.

While conventional prime-boost regimens using viral- or Listeria-based boosters can induce high frequency cytotoxic T-lymphocyte (CTL) responses in the subject, these conventional prime-boost regimens also induce strong systemic innate immune responses that protect against antigen challenge (Liehl et al.). In contrast, the gene gun methods described herein and incorporated by reference herein (e.g., vaccination with plasmids that produce adjuvants but no inserted antigen) do not induce strong systemic innate immune responses which could negatively impact challenge.

In some embodiments, routes of administration for the first composition and/or the second composition include, but are not limited to, oral, intravascular, and intradermal administration.

When administered according to one or more of the malaria vaccination regimens described herein, the first composition of one or more of the malaria vaccine compositions primes CD8+ T cells in the subject. Without intending to be bound by any particular theory, primed CD8+ T cells are thought to lead to an increased number of resident memory CD8+ T cells in the subject's liver (liver Trm). In some embodiments, following administration of one or more of the malaria vaccine compositions, a first number of liver Trm cells in the mammal's liver increases relative to a second number of Trm cells. For example, during one or more of the malaria vaccination regimens, such as following administration of the second composition of the malaria vaccine composition, the first number of liver Trm cells in the mammal's liver increases relative to the second number of Trm cells.

### Additional Associated Methods

The present technology additionally includes methods associated with the methods of vaccinating subjects with one or more of the malaria vaccine compositions and/or malaria vaccination regimens described above. These additional associated methods include, but are not limited to, methods of evaluating a subject's sera prior to administration of the one or more of the malaria vaccine compositions for reactivity against one or more sporozoites (e.g., pre-immunization sera). By evaluating pre-immunization sera, it is thought that a baseline titer below which the malaria vaccine compositions, regimens, and/or methods may be effective in populations of subjects, such as endemic populations. By identifying one or more baseline titers, a plurality of time periods can be identified by which to begin administering one or more of the malarial vaccine regimens and/or methods of the present technology to the subject. In some embodiments, if the baseline titer is achieved in childhood or at the end of a non-transmission season for malaria, then one or more time periods for vaccination can be identified. Baseline titers can also be combined with a seasonality of malaria to identify one or more time periods for vaccination at or near the relative nadir of sporozoite-specific antibody immunity. In some embodiments, the subject's humoral response can be induced following administration of one or more doses of the malaria vaccine compositions. Without intending to be bound by any particular theory, it is thought that determining one or more baseline titers and/or one or more time periods for vaccination that are optionally at or near the relative nadir maximizes formation of Trm cells in the subject's liver. It is further thought that subsequent vaccinations could be advantageous to the subject's immunity against malaria as formation of liver Trm would not be adversely affected by an antibody response.

Methods associated with the present technology also include one or more systems for testing whether a subject is protected against a potential malaria infection using one or more T cell antigens. These methods include challenging the subject with one or more sporozoites during an effector cytotoxic T-cell response (e.g., the peak of the response).

The following examples are intended to illustrate various embodiments of the invention. As such, the specific embodiments discussed are not to be construed as limitations on the scope of the invention. It will be apparent to one skilled in the art that various equivalents, changes, and modifications may be made without departing from the scope of invention, and it is understood that such equivalent embodiments are to be included herein. For example, although the Examples describe studies related to aptamer-shRNA fusions using aptamers to HIV integrase, one skilled in the art would understand that any aptamer may be fused to any applicable shRNA molecule and/or miRNA molecule based on the methods described below in order to bind to, reduce activity of, and/or target for degradation, a protein in a target cell. Non-limiting examples of aptamers, shRNA molecules, and miRNA molecules that may be used are described above. Further, all references cited in the disclosure are hereby incorporated by reference in their entirety, as if fully set forth herein.

### EXAMPLES

### Example 1: Prime and Trap T-Cell Approach for Murine Malaria Parasites

This example describes studies where the prime and trap approach discussed herein was used on murine subjects having one or more malaria parasites.

### Materials and Methods

Mice: Female BALB/cj mice (4-6 wk old) were obtained from Jackson Laboratories (Barr Harbor, Me.), housed in an IACUC-approved animal facility at the University of Washington and used under an approved IACUC protocol.

DNA vaccination: PyCSP minigene vaccines encoding a 33-amino acid segment containing a partial CSP fragment centered on the epitope SYVPSAQI were constructed in the NTC vector (Nature Technology, Lincoln, Nebr.) and tagged with a N-terminal ubiquitin tag. An E. coli heat-labile toxin-encoding plasmid was used as an adjuvant (Arrington et al.). Purified DNA was loaded onto gold beads and mice were vaccinated using a PowderJect-style gene gun on trimmed abdominal skin by cluster priming (two cartridges on Days 0 and 2) and were booster where indicated 4 weeks later.

Sporozoite vaccination and challenge: A. stephensi mosquitoes infected with wild-type P. yoelii 17XNL and P. yoelii fabb/f^{-/-} (GAP) were reared at the CID Research facility. Sporozoites were obtained by salivary gland dissection and gradient purification (Kennedy et al.). Radiation-attenuated sporozoites (RAS) were generated from WT Py by x-ray exposure (10,000 rads; Rad Source, Suwanee, Ga.). Sporozoites were administered intravenously in a volume of 100-150 μL.

Liver lymphocyte flow cytometry: Liver lymphocytes were isolated by mechanical dissociation and Percoll density gradient adapted from Blom et al. Briefly, livers were perfused with 10 mL PBS with 2 mM EDTA by injection into the portal vein draining from the inferior vena cava. Gull bladder was removed, and livers were placed in 5 mL RPMI supplemented with glutamine with 5% FBS (RP5) on ice to ensure cell survival. Livers were mashed through a 200 μm (Pluriselect 435020003) mesh filter with the back of a 3 mL syringe plunger. Mesh filter and plunger were washed with 40 mL RP5. The cell suspension was spun at 600 rpm for 1 min at 4° C. without brake, supernatants were collected and moved to a clean 50 mL conical where they were spun at 1500 rpm for 8 min at 4° C. The cell pellet was resuspended in 10 mL room temperature 35% Percoll (GE Health Sciences) in HBSS supplemented with 100 U heparin and spun at room temperature at 2000 rpm for 25 min without break. Final cell pellet containing IHLs were resuspended in 2 mL ACK lysis buffer for 2-3 min and quenched with 8 mL 1×MACs buffer (PBS, 1 mM EDTA, 0.5% FBS) and then spun at 1400 rpm at 4 C for 5 min. Final pellets were resuspended in 100 μL 1×MACs buffer and moved to a 96-well plate for blocking (30 min), antibody staining (45 min), fixing (20 min), and analyzing by flow cytometry.

The following antibodies were used to assess liver resident memory cells: CD3e-BUV395 (clone 145-2C11, BD), B220-BV711 (clone RA3-6B2, BioLegend), CD4-Alexa fluor 700 (clone GK1.5, BioLegend), CD8a-BV421 (clone 53-607, BD), CD69-BV510 (clone H1.2F3, BD), CD44-Alexa fluor 488 (clone IM7, BioLegend), CD62L-PE-Cy7, BD), KLRG1-PerCP-Cy5.5, BioLegend), CXCR6-PE (clone 221002, R&D Systems), CSP-tetramer (NIH Tetramer Core) conjugated to streptavidin-APC (ProZyme) per standard protocols, blocking before staining was done with Fc CD16/32 (clone 2.4G2, BD). Cells were gated for CD8⁺ T cells (CD3e⁺, B220⁻, CD4⁻), CD44^{hi} by CD62^{lo}, then assessed by either KLRG1^{lo} by CD69^{hi} or by CXCR6^{hi} by CD69^{hi}. Antigen specificity was then assessed by CSP-tetramer. Cell count per gram of tissue was calculated based on a known concentration of counting beads per samples (Polyscience 18328-5) to normalize data.

Ex vivo IFNγELISPOT: For ELISPOTs, peptides (1 μg/mL final) were combined with 1×10⁶ murine splenocytes by murine interferon-γ (IFNγ) ELISPOT (eBioscience) and cultured 18 hr at 37° C. as reported (Murphy et al.). For ELISPOTs from Lm experiments, 1×10⁵ splenocytes from sensitized animals were combined with 5×10⁵ naïve splenocytes per well.

Efficacy assessments: In some experiments, luciferase-based in vivo imaging of liver burden or liver RNA extraction and Plasmodium 18S rRNA RT-PCR were performed as described (Billman et al.). In other experiments, thin blood smears were taken by tail vein bleeds, stained with Giemsa and evaluated for patent parasitemia.

Statistics: ELISPOT comparisons were by the unpaired, two-tailed Student's t-test. All comparisons of flow cytometry cell counts and liver burden (RT-PCR and IVIS) were by the non-parametric Mann-Whitney test. Protection was evaluated using Kaplan-Meier curves.

### Results

### DNA-Only CD8⁺ T Cell CSP Vaccine Fails to Protect at a Memory Time Point

To test whether the DNA-only, gene gun-delivered PyCSP DNA vaccines could induce T cell responses that protect against malaria antigen challenge, BALB/c mice were immunized with a DNA vaccine encoding the canonical H2-K^{d}-restricted PyCSP epitope SYVPSAEQI. This DNA vaccine formulation linked a CSP T cell epitope to an ubiquitin tag and does not induce CSP-specific antibody responses (data not shown). High frequency CSP-specific, IFNγ-producing CD8⁺ T cells could be detected in peripheral blood one month after vaccination prior to antigen challenge (3-15% of total CD8s) (FIG. 1A). However, these mice were unprotected against challenge with 250 Py wild-type sporozoites at memory time points with no significant difference in time to blood smear positivity compared to completely naïve control mice (FIG. 1B). This data suggested that either CSP-specific T cells were a non-protective phenotype, at an inadequate frequency at a memory time point to protect, and/or in the wrong compartment.

### DNA-Only Vaccines can Overcome the Lack of Trm at High Effector T Cell Frequencies

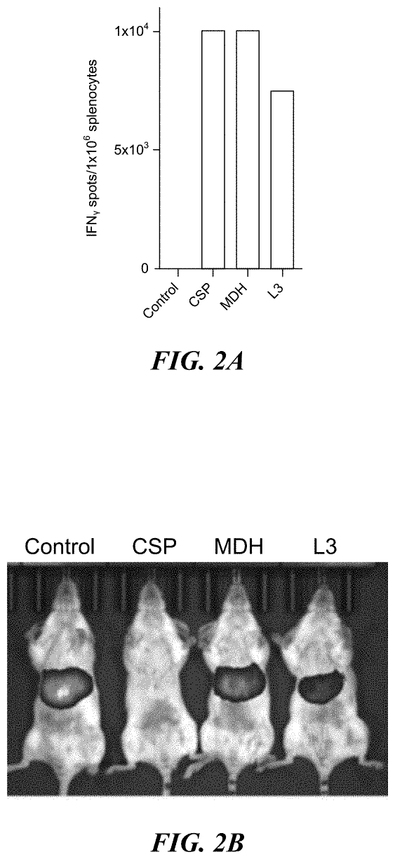

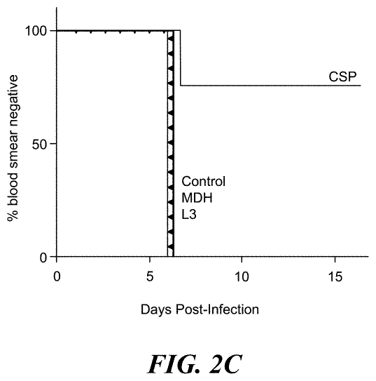

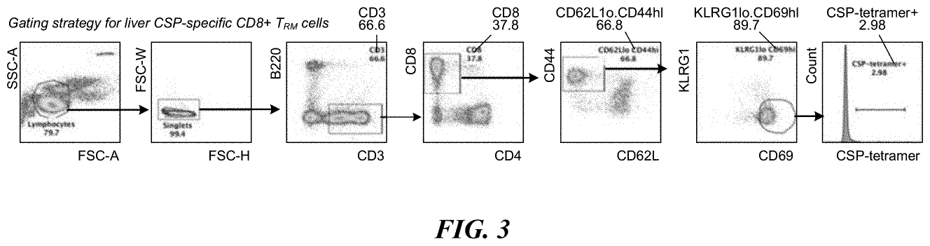

To begin to test these possibilities, mice were challenged at the peak of a DNA prime/DNA boost T cell response to determine if higher frequency effector CD8+ T cells were protective. DNA-only vaccines induced CD8+ T cell responses against PyCSP, PyL3 and PyMDH, three proteins with known H2-K^{d} epitopes where responses could be monitored using epitope-specific IFNg ELISPOTs (FIG. 2A). However, when DNA vaccinated mice were challenged with sporozoites five days after the DNA booster vaccination, non-protective antigens (e.g., L3, MDH) conferred no protection against acute challenge (FIG. 2B-C). In contrast, CSP DNA-vaccinated mice showed reductions in Py liver burden following a 5×10⁴ Pyluc challenge (FIG. 2A) and were sterilely protected against a 5×10² wild-type Py challenge measured by daily blood smears (FIG. 2C). CD8⁺ Trm cells can be identified by surface markers KLRG1^{lo} or CXCR6⁺ (not shown) and CD69⁺ (FIG. 3). These data show that the T cells targeting CSP are capable of providing sterile protection at high T cell frequencies following a DNA boost, but fail to provide sterile protection at later memory time points (i.e. weeks after the final DNA boost).

With this acute challenge model, immunization, challenge and efficacy endpoints can be assessed in <50 days after initiating the experiment. This data indicates that DNA-induced T cells have a functionally-appropriate phenotype to kill infected hepatocytes. Without intending to be bound by any particular theory, it is thought that such killing occurs through CTLs because the CSP minigene vaccine does not induce non-CD8-mediated immunity. These data suggest that DNA vaccines are capable of triggering phenotypically protective T cell responses.

### DNA-Only Vaccines Induce Liver Trm if Antigen-Encoding DNA is Expressed De Novo in the Liver

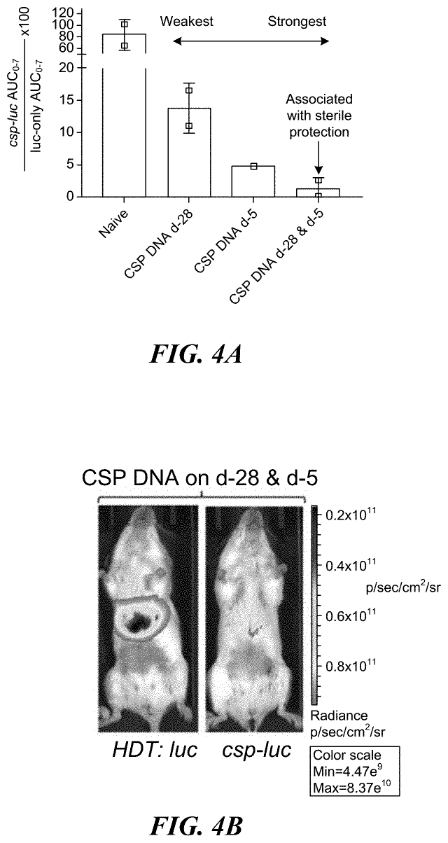

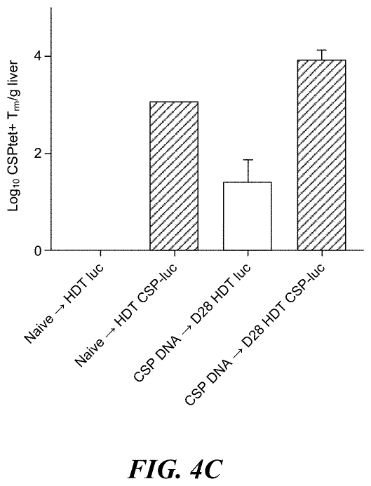

To investigate whether DNA-primed Trm were formed in the liver, performed hydrodynamic transfections (HDT) were performed. In HDT, approximately 10% body weight of DNA-containing saline was injected into the tail vein of an anesthetized mouse over 5-10 seconds. (Herweijer and Wolff; Kovacsics and Raper). The sudden increase in the oncotic pressure gradient drives injected plasmid DNA into hepatocytes, where plasmids are expressed. HDT plasmids were constructed to co-express PyCSP and luciferase (csp-T2A-luc plasmid) or luciferase only as a control (luc plasmid). When HDT was performed in immunologically-naïve BALB/c mice, generally comparable luminescence was observed by IVIS live-animal imaging over the course of seven days for both plasmids (FIG. 4A). In addition, the rate of luminescence decay for luc-only HDT in CSP DNA-immunized mice was similar to that of naïve mice. However, when csp-luc HDT was performed in mice previously gene gun-primed and boosted with PyCSP DNA, luminescence was ablated (99.2% reduction by 3 days and 99.9% by 7 days, FIG. 4B), indicating rapid killing of CSP-expressing cells. CSP-specific clearance was slower for mice immunized once with CSP DNA (28 or 5 days prior to HDT), indicating that CSP-specific killing may be dependent on effector T cell frequencies. CSP DNA-primed/boosted, csp-luc HDT boosted mice showed ˜12×10³ CD8⁺ Trm per g of liver 28 days post-HDT booster, markedly higher than for naïve mice (FIG. 4C). These Trm densities exceeded those thought to be required for protection (Fernandez-Ruiz et al.). Thus, these studies indicated that novel DNA vaccination regimens with antigen expression in the liver (e.g., gene gun plus HDT) induce liver Trm.

### DNA-Only Versus Sporozoite-Only Vaccination Differ in Liver Trm Frequencies

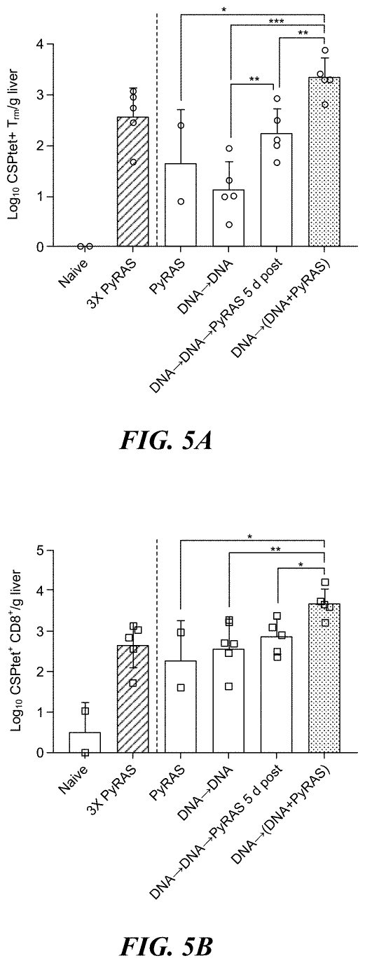

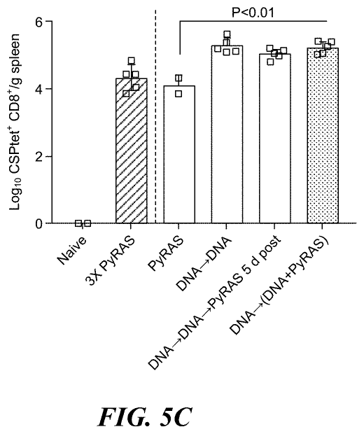

Mice were immunized with PyRAS one or more times or with gene gun-delivered PyCSP DNA twice. CSP-specific liver Trm were detected following repeated sporozoite immunization but not after DNA-only vaccination or after a single immunization with sporozoites (FIG. 5A). Total CSP-specific CD8+ T cells in the liver (FIG. 5B) and spleen (FIG. 5C) did not show this difference. DNA-only vaccination resulted in a higher frequency of CD8⁺ effectors in the spleen than sporozoites alone despite the lack of protection from DNA-only vaccination. Without intending to be bound by any particular theory, it is thought that the lack of protection in DNA-only vaccines may stem from poor formation of liver Trm.

### DNA Priming Followed by DNA-Plus-Sporozoite Boosting Induces High Frequency Liver Trm and Achieves Complete Protection Against Sporozoite Challenge

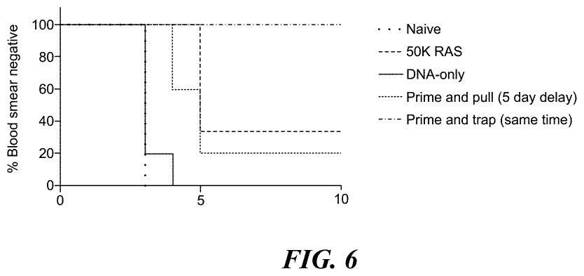

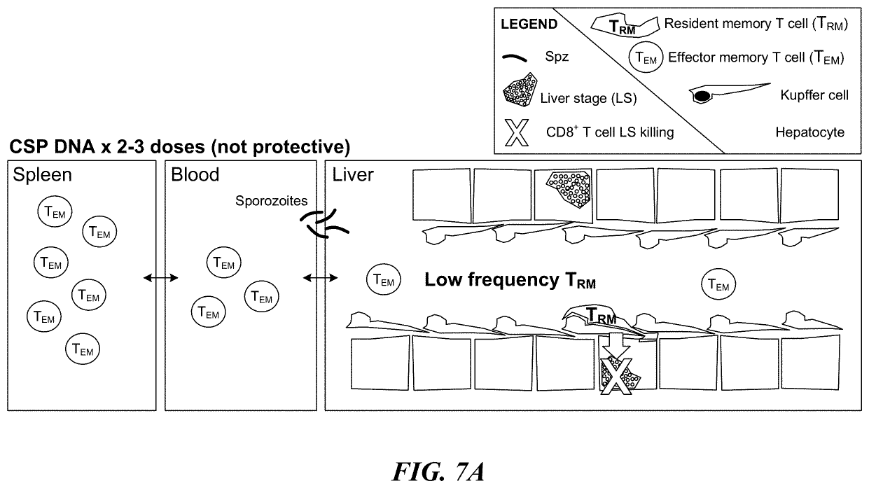

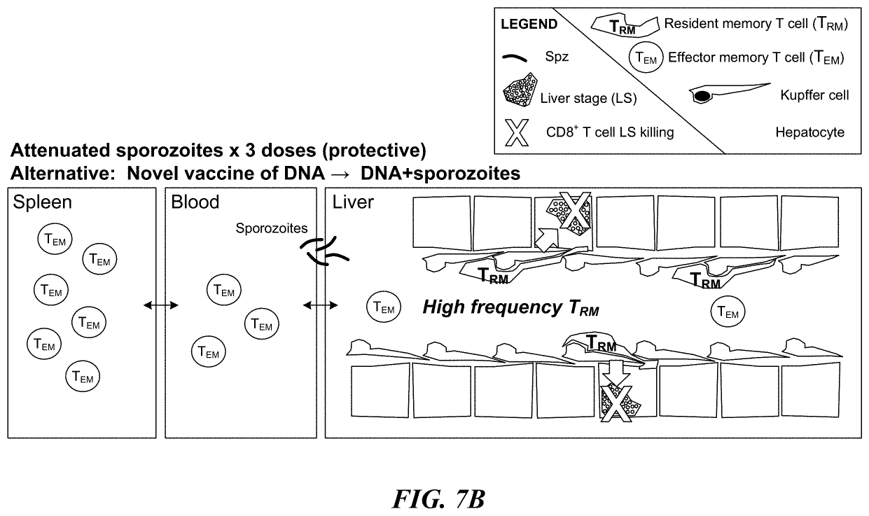

To combine T cell immunogenicity of the DNA vaccines described in Example 1 with the liver-specific targeting and Trm formation of sporozoites, mice were primed with CSP-specific DNA and boosted 4 weeks later with DNA combined with intravenously administered PyRAS. A delayed boost with PyRAS at five days after the DNA booster at a time corresponding with the peak of the DNA primed/boosted T cell response was also tested. The combination vaccine of gene gun DNA administered simultaneously with RAS (DNA→DNA+PyRAS) showed the highest liver Trm frequencies (FIG. 5A). Delaying the PyRAS dose until the peak of the DNA-induced response lowered liver Trm frequencies. When such mice were challenged with 500 wild-type sporozoites, the regimen of DNA priming followed by concurrent DNA-and-sporozoite boosting resulted in sterile protection, whereas other regimens were 0-20% protective (FIG. 6). This study provides a simplified two-dose vaccination regimen that increases CSP-specific liver Trm and provides complete protection in a mouse model of malaria vaccination (FIG. 7).

### Discussion

This Example is a proof-of-concept that a liver-targeted CD8⁺ T cell-mediated vaccine can be delivered using components suitable for use in humans. The data collectively show that gene gun-delivered DNA-only vaccines can prime high frequency CTL effector responses, but fail to produce protective liver Trm (FIG. 7A). In contrast, sporozoite vaccines induce liver Trm but achieve protective levels after multiple IV administrations (FIG. 7B). The prime-and-trap approach described herein includes aspects of both methods to utilize the increased precursor frequency of DNA-prime/boosted T cells and the liver-specific targeting of sporozoites (FIG. 7C).

The compositions, methods, and vaccine regimens described in this Example increase liver Trm and improve sterile protection in the murine model of malaria infection. As described herein, DNA priming utilizing vector/gene constructs that induce minimal antibody responses can be followed by a single, highly effective sporozoite-based boost for induction of protective liver-specific Trm. The use of sporozoites as a one-time booster may ease the workflow and delivery issues that have hampered enthusiasm for sporozoite hyperimmunization regimens.

The prime-and-trap approach can be easily and rapidly adapted for testing in non-human primates and/or human clinical trials. Both components (DNA and sporozoites) have been tested in numerous studies in humans. While CSP-specific CTL are protective in this animal model (Balam et al.; Bongfen et al.; Kumar et al.), CSP-only priming may be inadequate to achieve protection in humans due to MHC diversity. In addition, T cells that fail to reach the liver may not be able to contribute to protection as well as those that are pre-positioned in the liver (Spencer et al.). Combined with recent data (Fernandez-Ruiz et al.), this Example provides additional data suggesting why such high CSP-specific CTL frequencies were historically needed to achieve protection in mice (Balam et al.; Renia et al.; Rodrigues et al.; Romero et al.; Schmidt et al.; Schmidt et al.). Without intending to be bound by any particular theory, it is thought that these strategies did not induce CSP-specific liver Trm and therefore a high number of CD8⁺ T cells was needed to cross the protective threshold in the liver.

The DNA-only ‘acute challenge model’ described in this Example may also form a system for testing protection by novel T cell antigens.

If immunized mice could be challenged with sporozoites at the peak of the effector CTL response (day 6 in mice), protection could be rapidly assessed. While many prime-boost regimens with viral- or Listeria-based boosters can induce very high frequency CTL responses, they also induce strong systemic innate immune responses that alone are sufficient to protect against challenge (Liehl et al.). In contrast, gene gun vaccination with plasmids that produce adjuvants but no inserted antigen have no effect on challenge and do not raise the background in ELISPOTs conducted on lymphocytes from the spleen or liver.

### REFERENCES

Arrington, J., R. P. Braun, L. Dong, D. H. Fuller, M. D. Macklin, S. W. Umlauf, S. J. Wagner, M. S. Wu, L. G. Payne, and J. R. Haynes. 2002. Plasmid vectors encoding cholera toxin or the heat-labile enterotoxin from Escherichia coli are strong adjuvants for DNA vaccines. J Virol 76:4536-4546.
Balam, S., J. F. Romero, S. E. Bongfen, P. Guillaume, and G. Corradin. 2012. CSP-A Model for In Vivo Presentation of Plasmodium berghei Sporozoite Antigens by Hepatocytes. PloS one 7:e51875.
Bauza, K., E. Atcheson, T. Malinauskas, A. M. Blagborough, and A. Reyes-Sandoval. 2015. Tailoring a Combination Preerythrocytic Malaria Vaccine. Infect Immun 84:622-634.
Billman, Z. P., A. Kas, B. C. Stone, and S. C. Murphy. 2016a. Defining rules of CD8(+) T cell expansion against pre-erythrocytic Plasmodium antigens in sporozoite-immunized mice. Malar J 15:238.
Billman, Z. P., A. M. Seilie, and S. C. Murphy. 2016b. Purification of Plasmodium Sporozoites Enhances Parasite-Specific CD8+ T Cell Responses. Infect Immun 84:2233-2242.
Blom, K. G., M. R. Qazi, J. B. Matos, B. D. Nelson, J. W. DePierre, and M. Abedi-Valugerdi. 2009. Isolation of murine intrahepatic immune cells employing a modified procedure for mechanical disruption and functional characterization of the B, T and natural killer T cells obtained. Clin Exp Immunol 155:320-329.
Bongfen, S. E., R. Torgler, J. F. Romero, L. Renia, and G. Corradin. 2007. Plasmodium berghei-infected primary hepatocytes process and present the circumsporozoite protein to specific CD8+ T cells in vitro. J Immunol 178:7054-7063.
Chen, L., G. J. Keitany, X. Peng, C. Gibson, I. Mohar, M. Vignali, I. N. Crispe, F. Huang, and R. Wang. 2014. Identification of pre-erythrocytic malaria antigens that target hepatocytes for killing in vivo and contribute to protection elicited by whole-parasite vaccination. PloS one 9:e102225.
Doolan, D. L., and S. L. Hoffman. 2000. The complexity of protective immunity against liver-stage malaria. J Immunol 165:1453-1462.
Dunachie, S. J., M. Walther, J. E. Epstein, S. Keating, T. Berthoud, L. Andrews, R. F. Andersen, P. Bejon, N. Goonetilleke, I. Poulton, D. P. Webster, G. Butcher, K. Watkins, R. E. Sinden, G. L. Levine, T. L. Richie, J. Schneider, D. Kaslow, S. C. Gilbert, D. J. Carucci, and A. V. Hill. 2006. A DNA prime-modified vaccinia virus ankara boost vaccine encoding thrombospondin-related adhesion protein but not circumsporozoite protein partially protects healthy malaria-naive adults against Plasmodium falciparum sporozoite challenge. Infect Immun 74:5933-5942.
Fernandez-Ruiz, D., W. Y. Ng, L. E. Holz, J. Z. Ma, A. Zaid, Y. C. Wong, L. S. Lau, V. Mollard, A. Cozijnsen, N. Collins, J. Li, G. M. Davey, Y. Kato, S. Devi, R. Skandari, M. Pauley, J. H. Manton, D. I. Godfrey, A. Braun, S. S. Tay, P. S. Tan, D. G. Bowen, F. Koch-Nolte, B. Rissiek, F. R. Carbone, B. S. Crabb, M. Lahoud, I. A. Cockburn, S. N. Mueller, P. Bertolino, G. I. McFadden, I. Caminschi, and W. R. Heath. 2016. Liver-Resident Memory CD8+ T Cells Form a Front-Line Defense against Malaria Liver-Stage Infection. Immunity 45:889-902.
Gruner, A. C., M. Mauduit, R. Tewari, J. F. Romero, N. Depinay, M. Kayibanda, E. Lallemand, J. M. Chavatte, A. Crisanti, P. Sinnis, D. Mazier, G. Corradin, G. Snounou, and L. Renia. 2007. Sterile protection against malaria is independent of immune responses to the circumsporozoite protein. PloS one 2:e1371.
Haddad, D., E. Bilcikova, A. A. Witney, J. M. Carlton, C. E. White, P. L. Blair, R. Chattopadhyay, J. Russell, E. Abot, Y. Charoenvit, J. C. Aguiar, D. J. Carucci, and W. R. Weiss. 2004. Novel antigen identification method for discovery of protective malaria antigens by rapid testing of DNA vaccines encoding exons from the parasite genome. Infection and immunity 72:1594-1602.
Herweijer, H., and J. A. Wolff. 2007. Gene therapy progress and prospects: hydrodynamic gene delivery. Gene therapy 14:99-107.
Ishizuka, A. S., K. E. Lyke, A. DeZure, A. A. Berry, T. L. Richie, F. H. Mendoza, M. E. Enama, I. J. Gordon, L. J. Chang, U. N. Sarwar, K. L. Zephir, L. A. Holman, E. R. James, P. F. Billingsley, A. Gunasekera, S. Chakravarty, A. Manoj, M. Li, A. J. Ruben, T. Li, A. G. Eappen, R. E. Stafford, C. N. K, T. Murshedkar, H. DeCederfelt, S. H. Plummer, C. S. Hendel, L. Novik, P. J. Costner, J. G. Saunders, M. B. Laurens, C. V. Plowe, B. Flynn, W. R. Whalen, J. P. Todd, J. Noor, S. Rao, K. Sierra-Davidson, G. M. Lynn, J. E. Epstein, M. A. Kemp, G. A. Fahle, S. A. Mikolajczak, M. Fishbaugher, B. K. Sack, S. H. Kappe, S. A. Davidson, L. S. Garver, N. K. Bjorkstrom, M. C. Nason, B. S. Graham, M. Roederer, B. K. Sim, S. L. Hoffman, J. E. Ledgerwood, and R. A. Seder. 2016. Protection against malaria at 1 year and immune correlates following PfSPZ vaccination. Nat Med 22:614-623.
Keitany, G. J., B. Sack, H. Smithers, L. Chen, I. K. Jang, L. Sebastian, M. Gupta, D. N. Sather, M. Vignali, A. M. Vaughan, S. H. Kappe, and R. Wang. 2014. Immunization of mice with live-attenuated late liver stage-arresting Plasmodium yoelii parasites generates protective antibody responses to preerythrocytic stages of malaria. Infect Immun 82:5143-5153.
Kennedy, M., M. E. Fishbaugher, A. M. Vaughan, R. Patrapuvich, R. Boonhok, N. Yimamnuaychok, N. Rezakhani, P. Metzger, M. Ponpuak, J. Sattabongkot, S. H. Kappe, J. C. Hume, and S. E. Lindner. 2012. A rapid and scalable density gradient purification method for Plasmodium sporozoites. Malaria journal 11:421.
Kiniry, B. E., S. Li, A. Ganesh, P. W. Hunt, M. Somsouk, P. J. Skinner, S. G. Deeks, and B. L. Shacklett. 2017. Detection of HIV-1-specific gastrointestinal tissue resident CD8(+) T-cells in chronic infection. Mucosal Immunol
Kovacsics, D., and J. Raper. 2014. Transient expression of proteins by hydrodynamic gene delivery in mice. Journal of visualized experiments: JoVE
Kumar, K. A., P. Baxter, A. S. Tarun, S. H. Kappe, and V. Nussenzweig. 2009. Conserved protective mechanisms in radiation and genetically attenuated uis3(−) and uis4(−) Plasmodium sporozoites. PloS one 4:e4480.
Kumar, K. A., G. Sano, S. Boscardin, R. S. Nussenzweig, M. C. Nussenzweig, F. Zavala, and V. Nussenzweig. 2006. The circumsporozoite protein is an immunodominant protective antigen in irradiated sporozoites. Nature 444:937-940.
Lau, L. S., D. Fernandez Ruiz, G. M. Davey, T. F. de Koning-Ward, A. T. Papenfuss, F. R. Carbone, A. G. Brooks, B. S. Crabb, and W. R. Heath. 2011. Blood-stage Plasmodium berghei infection generates a potent, specific CD8+ T-cell response despite residence largely in cells lacking MHC I processing machinery. The Journal of infectious diseases 204:1989-1996.
Liehl, P., P. Meireles, I. S. Albuquerque, M. Pinkevych, F. Baptista, M. M. Mota, M. P. Davenport, and M. Prudencio. 2015. Innate immunity induced by Plasmodium liver infection inhibits malaria reinfections. Infection and immunity 83:1172-1180.
Limbach, K., J. Aguiar, K. Gowda, N. Patterson, E. Abot, M. Sedegah, J. Sacci, and T. Richie. 2011. Identification of two new protective pre-erythrocytic malaria vaccine antigen candidates. Malaria journal 10:65.
Longley, R. J., K. Bauza, K. J. Ewer, A. V. Hill, and A. J. Spencer. 2015. Development of an in vitro assay and demonstration of Plasmodium berghei liver-stage inhibition by TRAP-specific CD8+ T cells. PloS one 10:e0119880.
Lyke, K. E., A. S. Ishizuka, A. A. Berry, S. Chakravarty, A. DeZure, M. E. Enama, E. R. James, P. F. Billingsley, A. Gunasekera, A. Manoj, M. Li, A. J. Ruben, T. Li, A. G. Eappen, R. E. Stafford, N. Kc, T. Murshedkar, F. H. Mendoza, I. J. Gordon, K. L. Zephir, L. A. Holman, S. H. Plummer, C. S. Hendel, L. Novik, P. J. Costner, J. G. Saunders, N. M. Berkowitz, B. J. Flynn, M. C. Nason, L. S. Garver, M. B. Laurens, C. V. Plowe, T. L. Richie, B. S. Graham, M. Roederer, B. K. Sim, J. E. Ledgerwood, S. L. Hoffman, and R. A. Seder. 2017. Attenuated PfSPZ Vaccine induces strain-transcending T cells and durable protection against heterologous controlled human malaria infection. Proc Natl Acad Sci USA 114:2711-2716.
Mauduit, M., A. C. Gruner, R. Tewari, N. Depinay, M. Kayibanda, J. M. Chavatte, J. F. Franetich, A. Crisanti, D. Mazier, G. Snounou, and L. Renia. 2009. A role for immune responses against non-CS components in the cross-species protection induced by immunization with irradiated malaria sporozoites. PloS one 4:e7717.
Mauduit, M., R. Tewari, N. Depinay, M. Kayibanda, E. Lallemand, J. M. Chavatte, G. Snounou, L. Renia, and A. C. Gruner. 2010. Minimal role for the circumsporozoite protein in the induction of sterile immunity by vaccination with live rodent malaria sporozoites. Infect Immun 78:2182-2188.
Mishra, S., U. Rai, T. Shiratsuchi, X. Li, Y. Vanloubbeeck, J. Cohen, R. S. Nussenzweig, E. A. Winzeler, M. Tsuji, and V. Nussenzweig. 2011. Identification of non-CSP antigens bearing CD8 epitopes in mice immunized with irradiated sporozoites. Vaccine 29:7335-7342.
Moorthy, V. S., S. McConkey, M. Roberts, P. Gothard, N. Arulanantham, P. Degano, J. Schneider, C. Hannan, M. Roy, S. C. Gilbert, T. E. Peto, and A. V. Hill. 2003. Safety of DNA and modified vaccinia virus Ankara vaccines against liver-stage P. falciparum malaria in non-immune volunteers. Vaccine 21:1995-2002.
Mordmuller, B., G. Surat, H. Lagler, S. Chakravarty, A. S. Ishizuka, A. Lalremruata, M. Gmeiner, J. J. Campo, M. Esen, A. J. Ruben, J. Held, C. L. Calle, J. B. Mengue, T. Gebru, J. Ibanez, M. Sulyok, E. R. James, P. F. Billingsley, K. C. Natasha, A. Manoj, T. Murshedkar, A. Gunasekera, A. G. Eappen, T. Li, R. E. Stafford, M. Li, P. L. Feigner, R. A. Seder, T. L. Richie, B. K. Sim, S. L. Hoffman, and P. G. Kremsner. 2017. Sterile protection against human malaria by chemoattenuated PfSPZ vaccine. Nature
Murphy, S. C., A. Kas, B. C. Stone, and M. J. Bevan. 2013. A T-cell response to a liver-stage Plasmodium antigen is not boosted by repeated sporozoite immunizations. Proc Natl Acad Sci USA 110:6055-6060.
Pavelko, K. D., M. P. Bell, S. M. Harrington, and H. Dong. 2017. B7-H1 Influences the Accumulation of Virus-Specific Tissue Resident Memory T Cells in the Central Nervous System. Front Immunol 8:1532.
Pearson, F. E., C. O'Mahony, A. C. Moore, and A. V. Hill. 2015. Induction of CD8(+) T cell responses and protective efficacy following microneedle-mediated delivery of a live adenovirus-vectored malaria vaccine. Vaccine 33:3248-3255.
Regules, J. A., J. F. Cummings, and C. F. Ockenhouse. 2011. The RTS,S vaccine candidate for malaria. Expert Rev Vaccines 10:589-599.
Renia, L., D. Grillot, M. Marussig, G. Corradin, F. Miltgen, P. H. Lambert, D. Mazier, and G. Del Giudice. 1993. Effector functions of circumsporozoite peptide-primed CD4+ T cell clones against Plasmodium yoelii liver stages. J Immunol 150:1471-1478.
Richie, T. L., Y. Charoenvit, R. Wang, J. E. Epstein, R. C. Hedstrom, S. Kumar, T. C. Luke, D. A. Freilich, J. C. Aguiar, J. B. Sacci, Jr., M. Sedegah, R. A. Nosek, Jr., P. De La Vega, M. P. Berzins, V. F. Majam, E. N. Abot, H. Ganeshan, N. O. Richie, J. G. Banania, M. F. Baraceros, T. G. Geter, R. Mere, L. Bebris, K. Limbach, B. W. Hickey, D. E. Lanar, J. Ng, M. Shi, P. M. Hobart, J. A. Norman, L. A. Soisson, M. R. Hollingdale, W. O. Rogers, D. L. Doolan, and S. L. Hoffman. 2012. Clinical trial in healthy malaria-naive adults to evaluate the safety, tolerability, immunogenicity and efficacy of MuStDO5, a five-gene, sporozoite/hepatic stage Plasmodium falciparum DNA vaccine combined with escalating dose human GM-CSF DNA. Hum Vaccin Immunother 8:1564-1584.
Rodrigues, M. M., A. S. Cordey, G. Arreaza, G. Corradin, P. Romero, J. L. Maryanski, R. S. Nussenzweig, and F. Zavala. 1991. CD8+ cytolytic T cell clones derived against the Plasmodium yoelii circumsporozoite protein protect against malaria. Int Immunol 3:579-585.
Romero, P., J. L. Maryanski, G. Corradin, R. S. Nussenzweig, V. Nussenzweig, and F. Zavala. 1989. Cloned cytotoxic T cells recognize an epitope in the circumsporozoite protein and protect against malaria. Nature 341:323-326.
Sauzet, J. P., B. L. Perlaza, K. Brahimi, P. Daubersies, and P. Druilhe. 2001. DNA immunization by Plasmodium falciparum liver-stage antigen 3 induces protection against Plasmodium yoelii sporozoite challenge. Infection and immunity 69:1202-1206.
Schmidt, N. W., N. S. Butler, V. P. Badovinac, and J. T. Harty. 2010. Extreme CD8 T cell requirements for anti-malarial liver-stage immunity following immunization with radiation attenuated sporozoites. PLoS Pathog 6:e1000998.
Schmidt, N. W., R. L. Podyminogin, N. S. Butler, V. P. Badovinac, B. J. Tucker, K. S. Bahjat, P. Lauer, A. Reyes-Sandoval, C. L. Hutchings, A. C. Moore, S. C. Gilbert, A. V. Hill, L. C. Bartholomay, and J. T. Harty. 2008. Memory CD8 T cell responses exceeding a large but definable threshold provide long-term immunity to malaria. Proc Natl Acad Sci USA 105:14017-14022.
Seder, R. A., L. J. Chang, M. E. Enama, K. L. Zephir, U. N. Sarwar, I. J. Gordon, L. A. Holman, E. R. James, P. F. Billingsley, A. Gunasekera, A. Richman, S. Chakravarty, A. Manoj, S. Velmurugan, M. Li, A. J. Ruben, T. Li, A. G. Eappen, R. E. Stafford, S. H. Plummer, C. S. Hendel, L. Novik, P. J. Costner, F. H. Mendoza, J. G. Saunders, M. C. Nason, J. H. Richardson, J. Murphy, S. A. Davidson, T. L. Richie, M. Sedegah, A. Sutamihardja, G. A. Fahle, K. E. Lyke, M. B. Laurens, M. Roederer, K. Tewari, J. E. Epstein, B. K. Sim, J. E. Ledgerwood, B. S. Graham, and S. L. Hoffman. 2013. Protection against malaria by intravenous immunization with a nonreplicating sporozoite vaccine. Science 341:1359-1365.
Spencer, A. J., R. J. Longley, A. Gola, M. Ulaszewska, T. Lambe, and A. V. Hill. 2017. The Threshold of Protection from Liver-Stage Malaria Relies on a Fine Balance between the Number of Infected Hepatocytes and Effector CD8(+) T Cells Present in the Liver. J Immunol 198:2006-2016.
Strutt, T. M., K. Dhume, C. M. Finn, J. H. Hwang, C. Castonguay, S. L. Swain, and K. K. McKinstry. 2017. IL-15 supports the generation of protective lung-resident memory CD4 T cells. Mucosal Immunol
Tan, H. X., A. K. Wheatley, R. Esterbauer, S. Jegaskanda, J. J. Glass, D. Masopust, R. De Rose, and S. J. Kent. 2017. Induction of vaginal-resident HIV-specific CD8 T cells with mucosal prime-boost immunization. Mucosal Immunol
Tse, S. W., I. A. Cockburn, H. Zhang, A. L. Scott, and F. Zavala. 2013. Unique transcriptional profile of liver-resident memory CD8+ T cells induced by immunization with malaria sporozoites. Genes Immun 14:302-309.
White, K. L., H. L. Snyder, and U. Krzych. 1996. MHC class I-dependent presentation of exoerythrocytic antigens to CD8+ T lymphocytes is required for protective immunity against Plasmodium berghei. J Immunol 156:3374-3381.
WHO. 2016. World Malaria Report 2016.
Zhang, Y., Y. Qi, J. Li, S. Liu, L. Hong, T. Lin, C. Long, and X. Z. Su. 2012. A new malaria antigen produces partial protection against Plasmodium yoelii challenge. Parasitology research 110:1337-1345.

This disclosure is not intended to be exhaustive or to limit the present technology to the precise forms disclosed herein. Although specific embodiments are disclosed herein for illustrative purposes, various equivalent modifications are possible without deviating from the present technology, as those of ordinary skill in the relevant art will recognize. In some cases, well-known structures and functions have not been shown and/or described in detail to avoid unnecessarily obscuring the description of the embodiments of the present technology. Although steps of methods may be presented herein in a particular order, in alternative embodiments the steps may have another suitable order. Similarly, certain embodiments of the present technology disclosed in the context of particular embodiments can be combined or eliminated in other embodiments. Furthermore, while advantages associated with certain embodiments may have been disclosed in the context of those embodiments, other embodiments can also exhibit such advantages, and not all embodiments need necessarily exhibit such advantages or other advantages disclosed herein to fall within the scope of the present technology. Accordingly, this disclosure and associated technology can encompass other embodiments not expressly shown and/or described herein.

Throughout this disclosure, the singular terms “a,” “an,” and “the” include plural referents unless the context clearly indicates otherwise. Similarly, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the terms “comprising” and the like are used throughout this disclosure to mean including at least the recited feature(s) such that any greater number of the same feature(s) and/or one or more additional types of features are not precluded. Reference herein to “one embodiment,” “an embodiment,” or similar formulations means that a particular feature of a composition, a composition, a method, or a characteristic described in connection with the embodiment can be included in at least one embodiment of the present technology. Thus, the appearances of such phrases or formulations herein are not necessarily all referring to the same embodiment. Furthermore, various particular features, compositions, methods, or characteristics may be combined in any suitable manner in one or more embodiments.

All references listed herein (e.g., patents, patent applications, non-patent literature, presentations, posters, abstracts, and the like) are incorporated by reference herein in their entirety. To the extent any materials incorporated by reference herein conflict with the present disclosure, the present disclosure controls.

From the foregoing, it will be appreciated that specific embodiments of the invention have been described herein for purposes of illustration, but that various modifications may be made without deviating from the scope of the invention. Accordingly, the invention is not limited except as by the appended claims.

What is claimed is:

1. A malarial vaccination regimen comprising:
(i) a first composition comprising an antigenic subunit component, and
(ii) a second composition comprising a liver-specific antigenic component.

2. The malarial vaccination regimen of claim 1, wherein the antigenic subunit component of (i) is selected from the group consisting of:
(a) a wild-type or an attenuated Plasmodium parasite
(b) a deoxyribonucleic acid (DNA) polynucleotide;
(c) a ribonucleic acid (RNA) polynucleotide;
(d) a protein or a polypeptide;
(e) a virally-vectored antigen;
(f) a virus-like particle delivered antigen;
(g) a fragment of any of (b), (c), (d), (e), or (f)
(h) a subunit of (e) or (f); and
(i) a combination of any of (a), (b), (c), (d), (e), (f), (g), or (h).

3. The malarial vaccination regimen of claim 2, wherein the liver-specific antigenic component of (ii) is selected from the group consisting of:
(a) a wild-type or an attenuated Plasmodium parasite
(b) a deoxyribonucleic acid (DNA) polynucleotide;
(c) a ribonucleic acid (RNA) polynucleotide;
(d) a protein or a polypeptide;
(e) a virally-vectored antigen;
(f) a virus-like particle delivered antigen;
(g) a fragment of any of (b), (c), (d), (e), or (f)
(h) a subunit of (e) or (f); and
(i) a combination of any of (a), (b), (c), (d), (e), (f), (g), or (h).

4. The malarial vaccination regimen of claim 3, wherein the antigenic subunit component of (i) and/or the liver-specific antigenic component of (ii) is the DNA polynucleotide of (b) or the RNA polynucleotide of (c), and encodes a polypeptide comprising a circumsporozoite (CSP) fragment and/or another Plasmodium protein; or wherein the antigenic subunit component of (i) and/or the liver-specific antigenic component of (ii) is the DNA polynucleotide of (b) or the RNA polynucleotide of (c), and encodes a polypeptide comprising a protein from another liver-tropic pathogen, wherein the another liver-tropic pathogen is hepatitis C virus.

5. The malarial vaccination regimen of claim 3, wherein the antigenic subunit component of (i) and/or the liver-specific antigenic component of (ii) is the protein or polypeptide of (d) and/or the virus-like particle delivered antigen of (f), and comprises a circumsporozoite (CSP) fragment and/or another Plasmodium protein; or wherein the antigenic subunit component of (i) and/or the liver-specific antigenic component of (ii) is the protein or polypeptide of (d) and/or the virus-like particle delivered antigen of (f), and comprises a protein from another liver-tropic pathogen, wherein the another liver tropic pathogen is hepatitis C virus.

6. The malarial vaccination regimen of claim 1, wherein the polypeptide includes a tag.

7. The malarial vaccination regimen of claim 1, wherein the first composition and/or second composition is administered to a mammal with an adjuvant.

8. The malarial vaccination regimen of claim 1, wherein the sporozoite component comprises one or more sporozoites selected from the group consisting of one or more Plasmodium species, one or more recombinant Plasmodium species or strains, and one or more sporozoite strains.

9. The malarial vaccination regimen of claim 1, wherein an antibody response to the first composition and/or the second composition is not induced in a mammal following administration of the first composition and/or the second composition.

10. The malarial vaccination regimen of claim 3, wherein the first composition primes CD8+ T cells.

11. The malarial vaccination regimen of claim 10, wherein, a first number of resident memory CD8+ T (liver Trm) cells in the mammal's liver increases to a second number of Trm cells of following administration of the second composition.

12. The malarial vaccination regimen of claim 3, wherein first composition is administered at least one day, at least two days; at least three days, at least four days, at least five days, at least six days, at least seven days, at least ten days, at least two weeks, at least three week, at least four weeks, at least six weeks, or at least eight weeks before the second composition is administered.

13. A method of vaccinating a mammal, comprising the steps of:
(i) administering a first composition comprising an antigenic subunit component to the mammal; and
(ii) administering a second composition comprising a liver-specific antigenic component to the mammal;
wherein the first and second compositions are not administered concurrently and wherein a number of resident memory T cells in the liver are increased following administration of the first and/or second compositions.

14. The method of claim 13, wherein the antigenic subunit component of (i) is selected from the group consisting of:
(a) a wild-type or an attenuated Plasmodium parasite
(b) a deoxyribonucleic acid (DNA) polynucleotide;
(c) a ribonucleic acid (RNA) polynucleotide;
(d) a protein or a polypeptide;
(e) a virally-vectored antigen;
(f) a virus-like particle delivered antigen;
(g) a fragment of any of (b), (c), (d), (e), or (f)
(h) a subunit of (e) or (f); and
(i) a combination of any of (a), (b), (c), (d), (e), (f), (g), or (h).

15. The method of claim 14, wherein the liver-specific antigenic component of (ii) is selected from the group consisting of:
(a) a wild-type or an attenuated Plasmodium parasite
(b) a deoxyribonucleic acid (DNA) polynucleotide;
(c) a ribonucleic acid (RNA) polynucleotide;
(d) a protein or a polypeptide;
(e) a virally-vectored antigen;
(f) a virus-like particle delivered antigen;
(g) a fragment of any of (b), (c), (d), (e), or (f)
(h) a subunit of (e) or (f); and
(i) a combination of any of (a), (b), (c), (d), (e), (f), (g), or (h).

16. The method of claim 15, wherein the antigenic subunit component of (i) and/or the liver-specific antigenic component of (ii) is the DNA polynucleotide of (b) or the RNA polynucleotide of (c), and encodes a polypeptide comprising a circumsporozoite (CSP) fragment and/or another Plasmodium protein; or wherein the antigenic subunit component of (i) and/or the liver-specific antigenic component of (ii) is the DNA polynucleotide of (b) or the RNA polynucleotide of (c), and encodes a polypeptide comprising a protein from another liver-tropic pathogen, wherein the another liver-tropic pathogen is a hepatitis C virus.

17. The method of claim 15, wherein the antigenic subunit component of (i) and/or the liver-specific antigenic component of (ii) is the protein or polypeptide of (d) and/or the virus-like particle delivered antigen of (f), and comprises a circumsporozoite (CSP) fragment and/or another Plasmodium protein; or wherein the antigenic subunit component of (i) and/or the liver-specific antigenic component of (ii) is the protein or polypeptide of (d) and/or the virus-like particle delivered antigen of (f), and comprises a protein from another liver-tropic pathogen, wherein the another liver-tropic pathogen is a hepatitis C virus.

18. The method of claim 13, wherein the polypeptide includes a tag.

19. The method of claim 13, wherein the first composition and/or second composition is administered to the mammal with an adjuvant.

20. The method of claim 13, wherein the sporozoite component comprises one or more sporozoites selected from the group consisting of one or more Plasmodium species, one or more recombinant Plasmodium species or strains, and one or more sporozoite strains.

21. The method of claim 13, wherein an antibody response to the first composition and/or the second composition is not induced in the mammal following administration of the first composition and/or the second composition.

22. The method of claim 13, wherein the first composition primes CD8+ T cells.

23. The method of claim 22 wherein a first number of resident memory CD8+ T (liver Trm) cells in the mammal's liver increases to a second number of Trm cells of following administration of the second composition.

24. The method of claim 15, wherein first composition is administered at least one day, at least two days; at least three days, at least four days, at least five days, at least six days, at least seven days, at least ten days, at least two weeks, at least three week, at least four weeks, at least six weeks, or at least eight weeks before the second composition is administered.
